(19) United States
(12) Patent Application Publication
(10) Pub. No.: US 2016/0000804 A1
(43) Pub. Date: Jan. 7, 2016

(54) INHIBITORS OF NA(V) 1.9 CHANNEL ACTIVITY AND USES THEREOF FOR TREATING PAIN

(71) Applicants: UNIVERSITE D'AIX-MARSEILLE, Marseille (FR); CENTRE NATIONAL DE LA RECHERCHE SCIENTIFIQUE, Paris (FR)

(72) Inventors: PATRICK DELMAS, REMOLLON (FR); MURIEL EDITH AMSALEM, VITROLLES (FR); FRANÇOISE MARIE-DOMINIQUE PADILLA, TRETS (FR)

(21) Appl. No.: 14/769,228

(22) PCT Filed: Feb. 21, 2014
(86) PCT No.: PCT/EP2014/053390

§ 371 Date: Aug. 20, 2015

(30) Foreign Application Priority Data
Feb. 22, 2013 (EP) 13305198.7

(51) Int. Cl. A61K 31/56; A61K 9/00; A61K 45/06

(52) U.S. Cl. CPC A61K 31/56; A61K 45/06; A61K 9/0019

(57) ABSTRACT

The present invention relates to novel compounds for use for preventing, alleviating or treating pain in a subject. Also herein described are pharmaceutical compositions, their preparation and uses as well as methods for preventing, alleviating or treating pain using such compounds and compositions.

The present invention relates to novel compounds for use for preventing, alleviating or treating pain in a subject. Also herein described are pharmaceutical compositions, their preparation and uses as well as methods for preventing, alleviating or treating pain using such compounds and compositions.

### TECHNICAL BACKGROUND

Pain is the main reason for visiting the emergency department in more than 50% of cases (Cordell W H, Keene K K, Giles B K, Jones J B, Jones J H, Brizendine E J. The high prevalence of pain in emergency medical care. American Journal of Emergency Medicine. 2002; 20(3):165-9.) and is present in 30% of family practice visits (Hasselström J, Liu-Palmgren J, Rasjö-Wrååk G. Prevalence of pain in general practice. Eur J Pain. 2002; 6(5):375-85.). Several epidemiological studies from different countries have reported widely varying prevalence rates for chronic pain, ranging from 12-80% of the population (Abu-Saad Huijer H. Chronic pain: a review. J Med Liban. 2010; 58(1):21-7). It becomes more common as people approach death. A study of 4,703 patients found that 26% had pain in the last two years of life, increasing to 46% in the last month (Smith A K, Cenzer I S, Knight S J, Puntillo K A, Widera E, Williams B A, Boscardin W J, Covinsky K E. The epidemiology of pain during the last 2 years of life. Ann. Intern. Med. 2010; 153(9):563-9.).

Treatment of pain includes the use of local anesthetics, which block neuronal transmission and affect sensation as well as pain, and analgesics, which relieve pain and additionally may interfere with the activity of chemical mediators of inflammation.

Acute pain is usually managed with medications such as analgesics and anesthetics. Management of chronic pain, however, is much more difficult and may require the coordinated efforts of a pain management team, which typically includes medical practitioners, clinical psychologists, physiotherapists, occupational therapists, physician assistants, and nurse practitioners (Thienhaus, O; Cole, B E (2002). “The classification of pain”. In Weiner, R S. Pain management: A practical guide for clinicians. American Academy of Pain Management. p. 29. ISBN 0-8493-0926-3.).

An analgesic (also known as a painkiller) is any member of the group of drugs used to relieve pain (achieve analgesia). Their effectiveness relies on how they are able to block the nerve messages that are sent by the pain receptors to the brain. They further have an effect on the body temperature to increase it (known as fever) or to decrease it (Neuss, G. (2007). Chemistry: Course Companion. Oxford: Oxford University Press. p. 628).

Analgesic drugs act in various ways on the peripheral and central nervous systems; they include paracetamol (para-acetylaminophenol, also known in the US as acetaminophen or simply APAP), the non-steroidal anti-inflammatory drugs (NSAIDs) such as the salicylates, and opioid drugs such as morphine and opium. They are distinct from anesthetics, which reversibly eliminate sensation.

In choosing analgesics, the severity and response to other medication determines the choice of agent; the World Health Organization (WHO) pain ladder, originally developed in cancer-related pain, is widely applied to find suitable drugs in a stepwise manner (Anonymous (1990). Cancer pain relief and palliative care; report of a WHO expert committee. World Health Organization Technical Report Series, 804. Geneva, Switzerland: World Health Organization. pp. 1-75). It is a guideline on how pain should be managed. The first step contains the mild analgesics, such as aspirin, ibuprofen or paracetamol. If the pain persists or increases, then the patients should be treated with analgesics in the second step, which are weak opioids like codeine. If the pain still persists, then strong opioids should be used, such as morphine (Neuss, G. (2007). Chemistry: Course Companion. Oxford: Oxford University Press. pp. 628). The analgesic choice is also determined by the type of pain: for neuropathic pain, traditional analgesics are less effective, and there is often benefit from classes of drugs that are not normally considered analgesics, such as tricyclic antidepressants and anticonvulsants (Dworkin R H, Backonja M, Rowbotham M C, et al. (2003). “Advances in neuropathic pain: diagnosis, mechanisms, and treatment recommendations”. Arch. Neurol. 60 (11): 1524-34. Arch. Neurol. 60 (11): 1524-34.).

The exact mechanism of action of paracetamol/acetaminophen is uncertain, but it appears to be acting centrally rather than peripherally (in the brain rather than in nerve endings). Aspirin and the other non-steroidal anti-inflammatory drugs (NSAIDs) inhibit cyclooxygenases, leading to a decrease in prostaglandin production. This reduces pain and also inflammation (in contrast to paracetamol and the opioids).

Paracetamol has few side effects and is regarded as safe, although intake above the recommended dose can lead to liver damage, which can be severe and life-threatening, and occasionally kidney damage. While paracetamol is usually taken orally or rectally, an intravenous preparation introduced in 2002 has been shown to improve pain relief and reduce opioid consumption in the perioperative setting.

NSAIDs predispose to peptic ulcers, renal failure, allergic reactions, and occasionally hearing loss, and they can increase the risk of hemorrhage by affecting platelet function. The use of aspirin in children under 16 suffering from viral illness has been linked to Reye's syndrome, a rare but severe liver disorder.

Morphine, the archetypal opioid, and various other substances (e.g. codeine, oxycodone, hydrocodone, dihydromorphine, pethidine) all exert a similar influence on the cerebral opioid receptor system. Buprenorphine is thought to be a partial agonist of the opioid receptor, and tramadol is an opiate agonist with SNRI (Serotonin-Norepinephrine Reuptake Inhibitor) properties. Tramadol is structurally closer to venlafaxine than to codeine and delivers analgesia by not only delivering “opiate-like” effects (through mild agonism of the mu receptor) but also by acting as a weak but fast-acting serotonin releasing agent and norepinephrine reuptake inhibitor (Driessen B, Reimann W (January 1992). “Interaction of the central analgesic, tramadol, with the uptake and release of 5-hydroxytryptamine in the rat brain in vitro”. British Journal of Pharmacology 105 (1): 147-51.). Dosing of all opioids may be limited by opioid toxicity (confusion, respiratory depression, myoclonic jerks and pinpoint pupils), seizures (tramadol), but there is no dose ceiling in patients who accumulate tolerance. EP 0 300 806 describes a liposomal drug delivery and release system for the controlled release of butorphanol (an opioid analgesic).

Flupirtine is a centrally acting K⁺ channel opener with weak NMDA antagonist properties (Kornhuber J, Bleich S, Wiltfang J, Maler M, Parsons C G (1999). “Flupirtine shows functional NMDA receptor antagonism by enhancing Mg²⁺ block via activation of voltage independent potassium channels. Rapid communication”. J Neural Transm 106 (9-10): 857-67.) It is used in Europe for moderate to strong pain and migraine and its muscle relaxant properties. It has no anticholinergic properties and is believed to be devoid of any activity on dopamine, serotonin or histamine receptors. It is not addictive and tolerance usually does not develop (Klawe, C; Maschke, M (2009). “Flupirtine: pharmacology and clinical applications of a nonopioid analgesic and potentially neuroprotective compound”. Expert opinion on pharmacotherapy 10 (9): 1495-500.). However, tolerance may develop in single cases (Stoessel C, Heberlein A, Hillemacher T, Bleich S, Kornhuber J (August 2010). “Positive reinforcing effects of flupirtine-two case reports”. Prog. Neuropsychopharmacol. Biol. Psychiatry 34 (6): 1120-1.).

In patients with chronic or neuropathic pain, various other substances may have analgesic properties. Tricyclic antidepressants, especially amitriptyline, have been shown to improve treatment of pain in what appears to be a central manner. Nefopam is used in Europe for pain relief with concurrent opioids. The exact mechanism of carbamazepine, gabapentin and pregabalin is similarly unclear, but these anticonvulsants are used to treat neuropathic pain with differing degrees of success. Anticonvulsants are most commonly used for neuropathic pain as their mechanism of action tends to inhibit pain sensation.

Analgesics are frequently used in combination, such as the paracetamol and codeine preparations found in many non-prescription pain relievers. They can also be found in combination with vasoconstrictor drugs such as pseudoephedrine for sinus-related preparations, or with antihistamine drugs for allergy sufferers.

Anti-hyperalgesic is said of any compound that serves to counter hyperalgesia or that decrease sensitivity to pain. Hyperalgesia is an increased response to painful stimuli (pain amplification). SNRIs (Serotonin-norepinephrine reuptake inhibitors), NSAIDs (Non-steroidal anti-inflammatory drugs), glucocorticoid, opioids, gabapenton, and pregabalin are also identified as antihyperalgesic drugs.

While the use of paracetamol, aspirin, ibuprofen, naproxen and other NSAIDS concurrently with weak to mid-range opiates (up to about the hydrocodone level) has been said to show beneficial synergistic effects by combating pain at multiple sites of action (Mehlisch D R (2002). “The efficacy of combination analgesic therapy in relieving dental pain”. J Am Dent Assoc 133 (7): 861-71.), several combination analgesic products have been shown to have few efficacy benefits when compared to similar doses of their individual components. Moreover, these combination analgesics can often result in significant adverse events, including accidental overdoses, most often due to confusion which arises from the multiple (and often non-acting) components of these combinations (Murnion B. “Combination analgesics in adults”. Australian Prescriber (33): 113-5. http://www.australianprescriber.com/magazine/33/4/113/5. Retrieved 12 Aug. 2010).

U.S. Pat. No. 7,691,563 describe a putative use of sphingosine-lphosphate (SIP) to alleviate pain. SIP is a lipid second messenger produced from successive degradation of sphingomyelin into ceramide (upon sphingomyelinase enzyme activity), and further degradation of ceramide into SIP (upon ceramidase activity).

Inadequate treatment of pain is widespread throughout surgical wards, intensive care units, accident and emergency departments, in general practice, in the management of all forms of chronic pain including cancer pain, and in end of life care. This neglect is extended to all ages, from neonates to the frail elderly. African and Hispanic Americans are more likely than others to suffer needlessly in the hands of a physician; and women's pain is more likely to be undertreated than men's (Hoffmann D E, Tarzian A J. The girl who cried pain: a bias against women in the treatment of pain. J Law Med Ethics. 2001; 29(1):13-27. PMID 11521267). Improved treatments of pain are up to date still highly requested by patients in particular when considering inflammatory and/or chronic pains for which treatment remains incomplete whatever the selected known analgesic molecule.

### SUMMARY OF THE INVENTION

Inventors now herein provide a new solution to efficiently manage pain, for example acute pain, sub-acute pain, chronic pain, allodynia, hyperalgesia, partially treated pain, as well as refractory pains, while avoiding deleterious side effects, in particular gastrointestinal and renal deleterious side effects. In a preferred embodiment, the solution is to efficiently manage inflammatory pain, in particular inflammatory chronic pain; mechanical heat or cold sensitivity (or pain), typically hypersensitivity; visceral pain, in particular an enteric pain, more particularly a gastrointestinal pain or a pain associated with a gastrointestinal syndrome; and/or somatic pain, for example a neuralgia, in particular a trigeminal neuralgia.

This solution involves the use of a product inhibiting the Naᵥ1.9 channel activity, preferably a lipid inhibiting the Naᵥ1.9 channel activity and/or an inhibitor of the degradation of said lipid, for use as active(s) ingredient(s) for preventing, alleviating or treating pain in a subject.

The sodium channel, voltage-gated, type XI, alpha subunit also known as SCN11A, SCN12A, SNS2, or Naᵥ1.9 is a voltage-gated sodium ion channel protein which in humans is encoded by the SCN11A gene (HUGO Gene Nomenclature Committee ID=HGNC_ID:10583). Voltage-gated sodium channels are membrane protein complexes that play a fundamental role in the generation of action potentials and in the generation of subthreshold depolarization in excitable cells, such as neurons and muscle cells. The Nav1.9 α-subunit is selectively expressed in pain-transmitting neurons (e.g. nociceptors) in dorsal root ganglia and trigeminal ganglia. Its function is to sustain depolarizing drive and to generate tonic activity of action potentials in nociceptors. Strong expression is also reported in enteric neurons from the myenteric and sub-mucosal plexuses in the intestine. Alpha subunits, such as SCN11A, mediate voltage-dependent gating and ion transport, while auxiliary beta subunits regulate voltage-dependent properties of the channel and facilitate localization of the channel to the plasma membrane. Each alpha subunit consists of 4 domains connected by 3 intracellular loops; each domain consists of 6 transmembrane segments and intra- and extracellular linkers. Aberrant expression patterns, regulation or mutations of alpha subunits underlie a number of disorders.

When a lipid inhibiting the Naᵥ1.9 channel activity, i.e. the Naᵥ1.9 role in sustaining tonic firing of nociceptors and in the consecutive generation of heat and mechanical pain hypersensitivity, is used as an active ingredient for preventing, alleviating or treating pain in a subject, this lipid is preferably selected from cholesterol, a sphingolipid, preferably a sphingomyelin, and any combination thereof, and is even more preferably soluble cholesterol. Without wishing to be bound by a particular theory, inventors believe that such a lipid is capable of activating a new analgesic pathway by modulating ion channels controlling the excitability of nociceptive neurons.

Another solution involves the use, instead of the previously mentioned lipid or in addition to or in combination with said lipid, typically as an additional active compound, of an inhibitor of the degradation of a lipid inhibiting the Naᵥ1.9 channel activity, preferably of a compound selected from a cholesterol oxidase inhibitor, such as a morpholine derivate (Paul G. M. Hesselink, Antonius Kerkenaar, Bernard Witholt. (1990). Inhibition of microbial cholesterol oxidases by dimethylmorpholines. Journal of Steroid Biochemistry, 35(1) 107-113) or Cu²⁺ chelator clioquinol (Luigi Puglielli, Avi L. Friedlich, Kenneth D. R. Setchell, Seiichi Nagano, Carlos Opazo, Robert A. Cherny, Kevin J. Barnham, John D. Wade, Simon Melov, Dora M. Kovacs, and, Ashley I. Bush. (2005). Alzheimer disease β-amyloid activity mimics cholesterol oxidase J. Clinical Invest. 115(9) 2556-63), and/or a sphingomyelinase inhibitor, such as GW4869 (Luberto, C., Hassler, D. F., Signorelli, P., et al. (2002) “Inhibition of tumor necrosis factor-induced cell death in MCF7 by a novel inhibitor of neutral sphingomyelinase”. J Biol Chem 277(43) 41128-41139), or glutathione (Shin-ichi Yoshimura, Yoshiko Banno, Shigeru Nakashima, Katsuhiko Hayashi, Haruki Yamakawa, Motoshi Sawada, Noboru Sakai, Yoshinori Nozawa. (1999). Inhibition of Neutral Sphingomyelinase Activation and Ceramide Formation by Glutathione in Hypoxic PC12 Cell Death J. Neurochemistry 73(2)675-683).

In a particular embodiment, the product is a combination of a lipid inhibiting the Naᵥ1.9 channel activity and of an inhibitor of the degradation of a lipid inhibiting the Naᵥ1.9 channel activity.

The present invention further relates to i) a composition, typically a pharmaceutical composition, comprising at least such a product, typically a lipid inhibiting the Naᵥ1.9 channel activity and/or an inhibitor of the degradation of said lipid, as an active ingredient and, preferably a pharmaceutically acceptable carrier and/or at least one additional active distinct compound, as well as to ii) any uses thereof, in particular as a local analgesic or anti-hyperalgesic composition.

The composition can advantageously be formulated as a gel, a cream, an ointment, a slowly diffusing patch containing free lipids or lipids embedded in liposomal vesicles, or a liquid solution for intrathecal or intraspinal injection.

In a further aspect, methods for preventing, alleviating or treating pain in a subject are herein described. Typically, such a method comprises a step of administering to a subject in need thereof, or of exposing said subject, to a product or composition as herein described inhibiting the Naᵥ1.9 channel activity, possibly in combination with at least one other distinct active compound.

The present document further describes in vitro, in vivo, or ex vivo screening methods for identifying an agent that modulates a Naᵥ1.9 channel activity.

A particular method as herein described is an in vitro or ex vivo screening method for identifying an agent that modulates the Naᵥ1.9 channel activity, wherein said method comprises:

a) exposing or contacting a cell, typically an eukaryotic cell, preferably a neuron, expressing Naᵥ1.9 channels, to a test compound, and

b) detecting a decrease in response to a pain stimulus due to said exposition or contacting,

wherein said decrease identifies said test compound as an analgesic/antihyperalgic agent.

The decrease in response can be correlated to the electrical activity (electric potential) activated by Nav 1.9 channels (cf. FIG. 11) which can be measured using any one of the methods known by the skilled person as further described herein below.

A further herein described method is an in vitro or ex vivo screening method for identifying an agent that modulates the Naᵥ1.9 channel activity, comprising:

a) exposing or contacting a tissue comprising nerves and Naᵥ1.9 channels, for example a skin-nerve preparation or an artificial tissue, to a test compound, and

b) detecting a decrease in response to a pain stimulus due to said exposition or contacting,

wherein said decrease identifies said test compound as an analgesic/antihyperalgic agent.

The present document also describes an in vivo method for identifying an analgesic/antihyperalgic agent, comprising:

a) administering a test compound to an animal, in particular an agent having activity in the previously herein described methods, and

b) detecting in said animal a decrease in response to a pain stimulus due to said administering and

wherein said animal preferably retains mechanical sensitivity in physiological condition and preferably also the ability to respond to non-nociceptive sensory stimuli (typically hot, cold and touch),

wherein said decrease identifies said test compound as an analgesic/antihyperalgic agent.

This method can further comprise additional steps of administering a test compound to a Naᵥ1.9 KO animal and comparing the reaction (response to a pain stimulus) of aid KO animal to that of the wildtype littermate.

Another object of the invention is a kit comprising i) a product as herein described inhibiting the Naᵥ1.9 channel activity or a composition comprising such a product, preferably ii) at least one additional distinct active compound efficient against pain, and optionally iii) written instructions for using the kit. The present invention further covers the use of such a kit to prevent, alleviate or treat pain.

### DETAILED DESCRIPTION OF THE INVENTION

Pain is a well-known phenomenon as an indicator of actual or potential injury or tissue damage due to inflammation, ischemia, mechanical or other irritation. In pain science, thresholds are measured by gradually increasing the intensity of a stimulus such as electric current or heat applied to the body. The pain perception threshold is the point at which the stimulus begins to hurt, and the pain tolerance threshold is reached when the subject acts to stop the pain.

In 1994, responding to the need for a more useful system for describing chronic pain, the International Association for the Study of Pain (IASP) classified pain according to specific characteristics: (1) region of the body involved (e.g., abdomen, lower limbs), (2) system whose dysfunction may be causing the pain (e.g., nervous, gastrointestinal), (3) duration and pattern of occurrence, (4) intensity and time since onset, and (5) etiology (Merskey H & Bogduk N. Classification of Chronic Pain. 2 ed. Seattle: International Association for the Study of Pain; 1994, pp. 3-4). This system has been criticized by Clifford J. Woolf (Woolf C J, Bennett G J, Doherty M, Dubner R, Kidd B, Koltzenburg M, Lipton R, Loeser J D, Payne R, Torebjork E. Towards a mechanism-based classification of pain?. Pain. 1998; 77(3):227-9.) and others as inadequate for guiding research and treatment. According to Woolf (Woolf C J. What is this thing called pain?. Journal of Clinical Investigation. 2010; 120(11):3742-4.), there are three classes of pain : nociceptive pain, inflammatory pain which is associated with tissue damage and the infiltration of immune cells, and pathological pain which is a disease state caused by damage to the nervous system (neuropathic pain) or by its abnormal function (dysfunctional pain, like in fibromyalgia, irritable bowel syndrome, tension type headache, etc.).

Pain is usually transitory, lasting only until the noxious stimulus is removed or the underlying damage or pathology has healed, but some painful conditions, such as rheumatoid arthritis, peripheral neuropathy, cancer and idiopathic pain (pain that persists after the trauma or pathology has healed, or that arises without any apparent cause), may persist for years. Pain that lasts a long time is called chronic, and pain that resolves quickly is called acute. Traditionally, the distinction between acute and chronic pain has relied upon an arbitrary interval of time from onset; the two most commonly used markers being 3 months and 6 months since the onset of pain (Turk D C, Okifuji A. Pain terms and taxonomies of pain. In: Bonica J J, Loeser J D, Chapman C R, Turk D C, Butler S H. Bonica's management of pain. Hagerstown, Md.: Lippincott Williams & Wilkins; 2001.), though some theorists and researchers have placed the transition from acute to chronic pain at 12 months (Spanswick C C, Main C J. Pain management: an interdisciplinary approach. Edinburgh: Churchill Livingstone; 2000.). Others apply acute to pain that lasts less than 30 days, chronic to pain of more than six months duration, and subacute to pain that lasts from one to six months (Thienhaus O, Cole B E. Classification of pain. In: Weiner R. Pain management: a practical guide for clinicians. Boca Raton: CRC Press; 2002.). A popular alternative definition of chronic pain, involving no arbitrarily fixed durations is “pain that extends beyond the expected period of healing.” (Turk, D. C.; Okifuji, A. (2001). “Pain terms and taxonomies”. In Loeser, D.; Butler, S. H.; Chapman, J. J. et al. Bonica's management of pain (3 ed.). Lippincott Williams & Wilkins pp. 18-25. ISBN 0-683-30462-3.). Chronic pain may be classified as cancer pain or benign (Thienhaus, O.; Cole, B. E. (2002). “Classification of pain”. In Weiner, R. S. Pain management: A practical guide for clinicians (6 ed.). American Academy of Pain Management. ISBN 0-8493-0926-3.).

Nociceptive pain is caused by stimulation of peripheral nerve fibers that respond only to stimuli approaching or exceeding harmful intensity (nociceptors), and may be classified according to the mode of noxious stimulation; the most common categories being “thermal” (heat or cold), “mechanical” (crushing, tearing, etc.) and “chemical” (iodine in a cut, chili powder in the eyes).

Nociceptive pain may also be divided into “visceral”, “deep somatic” and “superficial somatic” pain. Visceral structures are highly sensitive to stretch, ischemia and inflammation, but relatively insensitive to other stimuli that normally evoke pain in other structures, such as burning and cutting.

Visceral pain is diffuse, difficult to locate and often referred to a distant, usually superficial, structure. It may be accompanied by nausea and vomiting and may be described as sickening, deep, squeezing, and dull. Deep somatic pain is initiated by stimulation of nociceptors in ligaments, tendons, bones, blood vessels, fasciae and muscles, and is dull, aching, poorly localized pain. Examples include sprains and broken bones. Superficial pain is initiated by activation of nociceptors in the skin or other superficial tissue, and is sharp, well-defined and clearly located. Examples of injuries that produce superficial somatic pain include minor wounds and minor (first degree) burns.

Neuropathic pain is caused by damage or disease affecting any part of the nervous system involved in bodily feelings (the somatosensory system) (Treede R D, Jensen T S, Campbell J N, Cruccu G, Dostrovsky J O, Griffin J W, Hansson P, Hughes R, Nurmikko T, Serra J. Neuropathic pain: redefinition and a grading system for clinical and research purposes. Neurology. 2008; 70(18):1630-5.). Peripheral neuropathic pain is often described as “burning,” “tingling,” “electrical,” “stabbing,” or “pins and needles” (Paice J A. Mechanisms and management of neuropathic pain in cancer. J. Support Oncol. 2003; 1(2): 107-20.)

Psychogenic pain, also called psychalgia or somatoform pain, is pain caused, increased, or prolonged by mental, emotional, or behavioral factors (“Psychogenic pain—definition from Biology-Online.org” Biology-online org. Retrieved Nov. 5, 2008). Headache, back pain, and stomach pain are sometimes diagnosed as psychogenic. Sufferers are often stigmatized, because both medical professionals and the general public tend to think that pain from a psychological source is not “real”. However, specialists consider that it is no less actual or hurtful than pain from any other source.

People with long term pain frequently display psychological disturbance, with elevated scores on the Minnesota Multiphasic Personality Inventory scales of hysteria, depression and hypochondriasis (the “neurotic triad”). Some investigators have argued that it is this neuroticism that causes acute injuries to turn chronic, but clinical evidence points the other way, to chronic pain causing neuroticism. When long term pain is relieved by therapeutic intervention, scores on the neurotic triad and anxiety fall, often to normal levels. Self-esteem, often low in chronic pain patients, also shows improvement once pain has resolved (Wall P D, Melzack R. The challenge of pain. New York: Penguin Books; 1996.).

Unless more precisely identified, the term “pain”, as used herein, indifferently refers to any pain or sensitivity (herein understood as an abnormal sensitivity, i.e. typically as an hypersensitivity), typically any Naᵥ1.9-mediated pain, in particular any pain selected from nociceptive pain, inflammatory pain, pathological pain, neuropathic pain, idiopathic pain, chronic pain, acute pain, subacute pain, thermal pain, mechanical pain, chemical pain, visceral pain, deep somatic pain, superficial somatic pain, somatoform pain, psychogenic pain, and psychalgia pain.

Inflammation is known to be responsible for the sensitization of peripheral sensory neurons, leading to spontaneous pain and invalidating pain hypersensitivity. Acute or chronic pathological tissue inflammation strongly impacts on pain perception by sensitizing peripheral sensory neurons, giving rise to local and incapacitating pain hypersensitivity. Inflammatory mediators are known to enhance nociceptive primary afferent fibers excitability, in part by modifying expression and/or function of ion channels present in nerve endings (Woolf C J, Costigan M (1999), Transcriptional and posttranslational plasticity and the generation of inflammatory pain. Proc Natl Acad Sci USA 96: 7723-7730).

Voltage-gated sodium channels (VGSCs) play a fundamental role in neuronal excitability as they are directly responsible for initiation and propagation of action potentials, and their implication in different chronic pain disorders, including inflammatory pain, is relatively well established (Lai J, Porreca F, Hunter J C, Gold M S (2004) Voltage-gated sodium channels and hyperalgesia. Annu Rev Pharmacol Toxicol 44: 371-397).

Among the 10 VGSC isoforms, two tetrodotoxin-resistant (TTX-R) channels, Nav1.8 and Nav1.9, are almost exclusively expressed in nociceptors, consistently with a specific involvement in nociceptive pathways (Akopian A N, Sivilotti L, Wood J N (1996) A tetrodotoxin-resistant voltage-gated sodium channel expressed by sensory neurons. Nature 379: 257-262; Persson A K, Black J A, Gasser A, Fischer T, Waxman S G (2010). Nociceptors are cells that act as receptors for painful stimuli, such as sensory neurons that are found in any area of the body that can sense noxious stimuli either externally or internally. Examples of external nociceptors are located in tissues such as skin (cutaneous nociceptors), cornea and mucosa. Internal nociceptors are located in a variety of organs, such as the muscle, joint, bladder, gut and continuing along the digestive tract. The cell bodies of these neurons are located in either the dorsal root ganglia or the trigeminal ganglia. The trigeminal ganglia are specialized nerves for the face, whereas the dorsal root ganglia associate with the rest of the body. The axons extend into the peripheral nervous system and terminate in branches to form receptive fields. In the enteric nervous system, Nav1.9 is detected within the soma and proximal axons of sensory, Dogiel type II, myenteric and submucosal neurons (Padilla et al. Expression and localization of the sodium channel in enteric neurons and in trigeminal sensory endings: Implication for intestinal reflex function and orofacial pain. Mol. Cell. Neurosci. 35 (2007) 138-152).

The Naᵥ1.9 channel, also herein identified as “Naᵥ1.9 channel” or “Naᵥ1.9”, is expressed in nociceptive dorsal root ganglion (“DRG”) neurons where it contributes to pain behavior after peripheral inflammation. Studies supported a role for the Naᵥ1.9 channel in contributing to pain behavior, in particular to spontaneous pain behavior, and to the persistent heat and mechanical hyperalgesia classically accompanying inflammation (Priest B T, Murphy B A, Lindia J A, Diaz C, Abbadie C, Ritter A M, Liberator P, Iyer L M, Kash S F, Kohler M G, Kaczorowski G J, MacIntyre D E, Martin W J. Contribution of the tetrodotoxin-resistant voltage-gated sodium channel NaV1.9 to sensory transmission and nociceptive behavior. Proc Natl Acad Sci USA. 2005, 102(26):9382-7; Amaya F, Wang H, Costigan M, Allchorne A J, Hatcher J P, Egerton J, Stean T, Morisset V, Grose D, Gunthorpe M J, Chessell I P, Tate S, Green P J, Woolf C J. The voltage-gated sodium channel Na(v)1.9 is an effector of peripheral inflammatory pain hypersensitivity. J Neurosci. 2006; 26(50):12852-60.). Inventors suggested that this Naᵥ1.9 channel may serve as molecular target for new type of analgesic with minimal side-effect profile (Padilla et al. Expression and localization of the sodium channel in enteric neurons and in trigeminal sensory endings: Implication for intestinal reflex function and orofacial pain. Mol. Cell. Neurosci. 35 (2007) 138-152). Maingret et al. (Inflammatory mediators increase Naᵥ1.9 current and excitability in nociceptors through a coincident detection mechanism. J Gen Physiol. (2008) 131(3), 211-225) demonstrated that Naᵥ1.9 channel is potentiated by the concerted action of inflammatory mediators that may contribute to nociceptor's hyperexcitability during peripheral inflammation.

More recently, this Naᵥ1.9 sodium channel has been characterized by inventors as not involved in basal pain thresholds but as crucial in the generation of heat and mechanical pain hypersensitivity, both in subacute and chronic inflammatory pain models, and to a lower extent in its maintenance (Lolignier et al., PLoS ONE, August 2011, Vol. 6, Issue 8, e23083).

Active Ingredient

An object of the present invention is a product inhibiting the Naᵥ1.9 channel activity for use as active ingredient for preventing, lessen, alleviating, making pain more bearable, or treating pain, in a subject, in particular any pain as herein described for example acute pain, sub-acute pain, chronic pain, allodynia, hyperalgesia, partially treated pain, as well as refractory pains, while preferably advantageously avoiding deleterious side effects, in particular gastrointestinal and renal deleterious side effects.

In a preferred embodiment, the product is to efficiently manage inflammatory pain, in particular inflammatory chronic pain; mechanical pain, in particular heat or cold sensitivity (or pain); visceral pain, in particular an enteric pain, for example a gastrointestinal pain or a pain associated with a gastrointestinal syndrome; and/or a somatic pain, for example a neuralgia, in particular a trigeminal neuralgia, for example an ocular pain.

The compounds according to the invention may also be used to prevent or treat chronic pain in subjects suffering from pathologies such as cancer, burns, etc., for which generally analgesics (such as morphine) may be administered for a long period, optionally in delayed form.

The compounds according to the invention may also be used together with reduced daily doses of morphine in order to improve the clinical picture of patients (by limiting side effects of morphinomimetics, such as intestinal disorders, for example).

The product of the invention is in addition advantageous in that it does not hide symptoms associated to the disease responsible for the pain affecting the subject, or associated to an aggravation of said disease, such as in particular fever.

In a particular embodiment, the product of the invention is a lipid inhibiting the Naᵥ1.9 canal activity for use as an active ingredient for preventing, alleviating or treating pain in a subject, in particular any pain as herein described.

The plasma membrane of cells is made of a combination of glycosphingolipids and protein receptors organized in glycolipoprotein microdomains termed lipid rafts (Levitan et al. (2010), review; Thomas S., Pais A. P., Casares S and Brumeanu T. D. (2004). Analysis of lipid rafts in T cells. Molecular Immunology 41: 399-409.). These specialized membrane microdomains compartmentalize cellular processes by serving as organizing centers for the assembly of signalling molecules, influencing membrane fluidity and membrane protein trafficking, and regulating neurotransmission and receptor trafficking (Korade, Z.; Kenworthy, A. K. (2008). “Lipid rafts, cholesterol, and the brain”. Neuropharmacology 55 (8): 1265.). Lipid rafts are more ordered and tightly packed than the surrounding bilayer, but float freely in the membrane bilayer (Simons, K.; Ehehalt, R. (2002). “Cholesterol, lipid rafts, and disease”. Journal of Clinical Investigation 110 (5): 597.). One key difference between lipid rafts and the plasma membranes from which they are derived is lipid composition. Research has shown that lipid rafts generally contain 3 to 5-fold the amount of cholesterol found in the surrounding bilayer. Also, lipid rafts are enriched in sphingomyelin, which is typically elevated by 50% compared to the plasma membrane.

Inventors now herein demonstrate that the Naᵥ1.9 channel is present in lipid rafts, in particular in lipid rafts from plasma membrane of DRG neurons. Inventors also discovered and herein demonstrate that a lipid depletion, preferably a cholesterol depletion, greatly enhances in vivo the Naᵥ1.9 channel activity. The lipid for use as an active ingredient in the context of the present invention is preferably selected from cholesterol, in particular soluble cholesterol, a sphingolipid, preferably a sphingomyelin, and any combination thereof.

In another embodiment, the active ingredient usable instead of the previously mentioned lipid, or in addition to or combination with, can be advantageously selected from inhibitors of cholesterol and/or of a sphingomyelin degrading enzymes. As previously detailed, an example of such a compound is the GW 4869 compound, which has been described by Luberto and coworkers (Luberto, C., Hassler, D. F., Signorelli, P., et al. (2002) “Inhibition of tumor necrosis factor-induced cell death in MCF7 by a novel inhibitor of neutral sphingomyelinase”. J Biol Chem 277(43) 41128-41139).

In a particular embodiment, the lipids of the invention, in particular cholesterol, or the inhibitor of cholesterol and/or sphingolipid degrading enzyme, in addition to Naᵥ1.9 advantageously modulate the activity of at least one other ion channel such as a potassium channel, calcium channel, ion co-transporter and the like, and/or affect the hERG channel or another physiologically relevant channel.

Subject

In the context of the present invention, the patient or subject is an animal, preferably a vertebrate, typically a mammal. In a preferred embodiment, the mammal is a human being, whatever its age or sex. The mammal may further be animal, in particular a domestic or breeding animal, in particular horses, dogs, etc.

In a particular embodiment, the subject suffers of an inflammatory pain, in particular a chronic inflammatory pain.

In another particular embodiment, the subject is resistant to the applied or to an already tested pain treatment, in particular to paracetamol, or suffers from gastrointestinal and/or renal adverse effects induced for example by aspirin or ibuprofen.

Compositions

A further object of the invention relates to a pharmaceutical composition comprising a product inhibiting the Naᵥ1.9 channel activity as herein described as an active ingredient, and preferably a pharmaceutically acceptable carrier.

A “pharmaceutical composition” refers to a formulation of a compound of the invention (active ingredient) and a medium generally accepted in the art for the delivery of biologically active compounds to the subject in need thereof. Such a carrier includes all pharmaceutically acceptable carriers, diluents, medium or supports therefore. This carrier can be selected for example from methyl-beta-cyclodextrin or other beta-cyclodextrin, a polymer of acrylic acid (such as carbopol), a mixture of polyethylene glycol and polypropylene glycol, monoethanol amine and hydroxymethyl cellulose.

When cholesterol is used as the active ingredient, such a carrier is only optional. When a soluble form of cholesterol is used, methylbetacyclodextrin for example can be used adequately in combination with cholesterol, as the only medium or together with another medium as previously described.

Conventional pharmaceutical practice may be employed to provide suitable formulations or compositions to subjects, for example in unit dosage form.

This composition is typically a local analgesic/anti-hyperalgesic composition.

The products of the invention (lipids, inhibitors of lipid degradation, and compositions) are for use for preventing, lessening, alleviating, making pain more bearable or treating pain in a subject. As explained previously, pain is typically selected from an acute pain; a subacute pain; and a chronic pain. Pain may further be selected from a mechanical sensitivity or pain, for example a heat or cold sensitivity or pain; an inflammatory pain; a visceral pain, in particular an enteric pain, more particularly a gastrointestinal pain or a pain associated with a gastrointestinal syndrome; allodynia; hyperalgesia; somatic pain, for example a neuralgia, in particular a trigeminal neuralgia (for example an ophthalmic pain); and any refractory pain.

Pain is preferably a chronic and/or inflammatory pain, even more preferably a chronic inflammatory pain.

In a particular embodiment, pain is a pain sensitive to steroidal anti-inflammatory drug (SAID), non steroidal anti-inflammatory drug (NSAID) or opioid, and said pain is to be treated with a combination of the product according to the invention together with a reduced amount of SAID, NSAID or opioid when compared to the administration of SAID, NSAID or opioid alone.

In another embodiment, pain is a pain where one of opioid, ibuprofen or aspirin is classically used and where doses are to be reduced to avoid their side effects, typically to avoid gastrointestinal and/or renal adverse effects.

In another particular embodiment, the subject or its pain itself is resistant to a drug selected from steroidal anti-inflammatory drug (SAID), non steroidal anti-inflammatory drug (NSAID) and opioid, or the subject suffers from gastrointestinal and/or renal adverse effects induced by said drug.

In a particular embodiment, pain is a pain resistant to paracetamol.

The composition of the invention can further comprise at least one additional active compound. This compound can be advantageously selected from a SAID, NSAID or opioid drug. In a preferred embodiment, the additional compound is a drug distinct from the product of the invention also treating or attenuating pain.

In another embodiment, a compound or composition of the invention can also be administered for example along with an agent intended to treat a coincident condition, such as where analgesic and antitumor agents are given together or contemporaneously.

Treatment

Also herein taught is a method for preventing, alleviating or treating pain in a subject. An aim of the method can be reducing the ion flux activity of the Naᵥ1.9 polypeptide.

A particular method for preventing, alleviating or treating a Naᵥ1.9-mediated pain in a subject in need thereof, comprises administering to the subject an effective amount of a product inhibiting the Naᵥ1.9 channel activity as herein described.

A further particular method for preventing, alleviating or treating pain, typically a Naᵥ1.9-mediated pain, in a subject in need thereof, comprises a step of administering to said subject, or of exposing said subject, to a compound or a composition as herein described inhibiting the Nav1.9 channel activity in a therapeutically effective amount, possibly in combination with at least one other active compound such as any one of the molecules mentioned in the background part, for example aspirin, ibuprofen, paracetamol, opioid, or such as an inhibitor of the degradation of a lipid inhibiting the Nav1.9 channel activity as herein described, preferably selected from a cholesterol oxidase inhibitor and a sphingomyelinase enzyme inhibitor.

In a further aspect, the present description relates to a method for treating a condition in a subject afflicted with a Nav1.9-mediated disease, such as but not limited to a pain as herein described, typically an inflammatory pain, in particular an inflammatory chronic pain, comprising administering to said subject an effective amount of a product of the invention.

As used in this specification “Naᵥ1.9-mediated disease” includes the wide range of disorders, conditions and disease that are caused by, or treatable with, modulation, preferably inhibition, of Naᵥ1.9 channel activity. Such diseases include but are not limited to pain (whether chronic, acute, inflammatory, etc.), neuralgia, neuropathic pain, eudynia, visceral pain, trauma pain, post-operative pain, heat sensitivity, irritable bowel syndrome, Crohn's disease, multiple sclerosis, diabetic neuropathy, arthritic pain, rheumatoid arthritis, sodium channel toxin related illnesses inducing pain, familial rectal pain, cancer and pain associated to chemotherapies, trigeminal neuralgia, migraine headache, and other headaches. In addition, Naᵥ1.9-mediated diseases include such pathologies as inflammatory diseases, neuropathies (e.g., diabetic neuropathy), dystrophies (e.g., reflex sympathetic dystrophy, post-herpetic neuralgia); and trauma (tissue damage by any cause). Inflammatory diseases can include, but are not limited to, visceral inflammatory pathologies and somatic inflammatory pathologies. Acute inflammatory pathologies include, but are not limited to proctite, rectite, and arthritis. Chronic inflammatory pathologies include, but are not limited to sarcoidosis, chronic inflammatory bowel disease, ulcerative colitis, and Crohn's pathology, psoriasis, rosacea, and vascular inflammatory pathologies, such as, but not limited to, disseminated intravascular coagulation, atherosclerosis, and Kawasaki's pathology. In addition, Naᵥ1.9-mediated disease also includes benign prostatic hyperplasia (BPH), hypercholesterolemia, cancer and pruritis. In particular, proctite, Crohn's pathology and ulcerative colitis are identified as visceral pains, arthritis, uveitis psoriasis and rosacea as somatic pains.

As used herein, “disease” includes disorder, condition, symptoms of a disease, incipient disease, anticipated disease or anticipated syndrome, and the like.

Protocol/Regimen

The compounds or compositions according to the invention may be administered in various ways or route. The product of the invention is preferably for cutaneous, subcutaneous, intrathecal, intra spinal, dermic, transdermic, ocular (for example corneal) or rectal administration to the subject, preferably for topic administration on an inflammation site.

In a preferred embodiment, the lipid of the invention, when used as the active ingredient of one of several active ingredients, is for topic administration, preferably through cutaneous, subcutaneous or transdermic administration to the subject, preferably for topic administration on an inflammation site.

A typical regimen for treatment of Naᵥ1.9-mediated disease comprises administration of an effective amount over a period of one or several days, up to one year, and including between one week and about six months, or it may be chronic. It is understood that the dosage of a pharmaceutical compound or composition of the invention administered in vivo will be dependent upon the age, health, sex, and weight of the recipient (subject), kind of concurrent treatment, if any, frequency of treatment, and the nature of the pharmaceutical effect desired. The ranges of effectives doses provided herein are not intended to be limiting and represent preferred dose ranges. However, the most preferred dosage will be tailored to the individual subject, as is understood and determinable by one skilled in the relevant arts (see, e.g., Berkowet et al., eds., The Merck Manual, 16^{th} edition, Merck and Co., Rahway, N.J., 1992; Goodmanetna., eds., Goodman and Cilman's The pharmacological Basis of Therapeutics, 10^{th} edition, Pergamon Press, Inc., Elmsford, N.Y., (2001); Avery's drug treatment: Principles and practice of clinical pharmacology and therapeutics, 3^{rd} edition, ADIS Press, LTD., Williams and Wilkins, Baltimore, Md. (1987), Ebadi, Pharmacology, Little, Brown and Co., Boston, (1985); Osolci et al., eds., Remington's Pharmaceutical Sciences, 18^{th} edition, Mack Publishing Co., Easton, Pa. (1990); Katzung, Basic and clinical pharmacology, Appleton and Lange, Norwalk, Conn. (1992)).

The total dose required for each treatment can be administered by multiple doses or in a single dose, preferably as soon as the early symptoms of pain appear, or preventively, for example before or during surgery when needed. The pharmaceutical compound can be administered alone or in conjunction with at least one other pharmaceutical directed to the pathology, or directed to other symptoms of the pathology. Effective amounts of a compound or composition according to the invention are from about 0.1 μg to about 100 mg/kg body weight, administered at intervals of 4-72 hours for a period of up to 1 year, and/or any range or value therein, such as 0.0001-1.0, 1-10, 10-50 and 50-100, 0.0001-0.001, 0.001-0.01, 0.01-0.1, 0.1-1.0, 1.0-10, 5-10, 10-20, 20-50, and 50-100 mg/kg, for example between 1 and 100 mg/kg, preferably between 1 and 5 mg/kg, for example 1,4, 1,5, 1,6, 1.7, 1,8 or 2 mg/kg, at intervals of 1-4, 4-10, 10-16, 16-24, 24-36, 36-48, 48-72 hours, for a period of 1-14, 14-28, or 30-44 days, or 1-24 weeks, or any range or value therein. The recipients of administration of compounds and/or compositions of the invention can be any subjects as herein defined, preferably humans.

Formulations/Concentrations

The compounds or compositions according to the invention may be administered in various forms. Thus, they may be formulated in the form of ointment, gel, paste, liquid solutions, suspensions, tablets, gelatin capsules, capsules, liposomes, suppository (in particular for pain associated with a gastrointestinal syndrome), powders, nasal drops, or aerosol, preferably in the form of ointment. Methods well known in the art for making formulations are found in, for example, Remington: The Science and Practice of Pharmacy, (19^{th} ed.) ed. A. R. Gennaro A R., 1995, Mack Publishing Company, Easton, Pa.

The compounds of the invention are typically administered in the form of ointments, gels, oils, tablets, suppositories, powders, gelatin capsules, capsules, etc., optionally by means of dosage forms or devices that ensure prolonged and/or delayed release. For this type of formulation, an agent such as cellulose, carbonate or starch is advantageously used.

For injections, the compounds are generally packaged in the form of liquid suspensions, which may be injected via syringes or perfusions, for example. In this respect, the compounds are generally dissolved in saline, physiological, isotonic or buffered solutions, etc., compatible with pharmaceutical use and known to the person skilled in the art. Thus, the compositions may contain one or more agents or excipients selected from dispersants, solubilizers, stabilizers, preservatives, etc. Agents or excipients that can be used in liquid and/or injectable formulations are notably methylcellulose, hydroxymethylcellulose, carboxymethylcellulose, polysorbate 80, mannitol, gelatin, lactose, vegetable oils, acacia, etc.

It is understood that the flow rate and/or dose administered may be adjusted by the person skilled in the art according to the patient, the pain observed, the area to be treated, the active product(s) concerned, the mode of administration, etc.

For topical applications, it is preferred to expose the subject to be treated to an effective amount of a pharmaceutical compound or composition according to the invention to target areas, e.g., skin surfaces, mucous membranes, and the like, which are adjacent to the peripheral neurons to be treated. Typically, the compounds are administered at doses that may vary between about 0.0001 mg to about 1 g/kg of body weight of a compound of the invention, more generally between about 1 and 100 mg/kg per application, depending upon the previously mentioned criteria, whether the use is prophylactic or therapeutic and the nature of the topical vehicle employed. A preferred topical preparation is an ointment or gel, wherein about 1 to about 100 mg/kg of active ingredient is used per cc (cm³) of ointment or gel base.

Furthermore, administration by injection may comprise several (2, 3 or 4) administrations per day, if need be.

In addition, for chronic treatments, delayed or prolonged systems may be advantageous, ensuring the subject effective and long-lasting pain treatment.

Screening

Further herein described are in vitro, in vivo, or ex vivo screening methods for identifying an agent that modulates a Naᵥ1.9 canal or channel (typically from mouse, rat or human origin) activity.

A particular method as herein described is an in vitro or ex vivo screening method for identifying an agent that modulates the Naᵥ1.9 channel activity, wherein said method comprises:

a) exposing or contacting a cell, typically an eukaryotic cell, preferably a neuron such as herein identified, expressing Naᵥ1.9 channels, to a test compound, and

b) detecting a decrease in response to a pain stimulus due to said exposition or contacting,

wherein said decrease identifies said test compound as an analgesic/antihyperalgic agent.

The decrease in response can be correlated to the electrical activity (electric potential) activated by Naᵥ1.9 channels (cf. FIG. 11) which can be measured using any one of the methods known by the skilled person as further described herein below.

The cell can be a cell naturally expressing the Naᵥ1.9 channel (endogenous expression) or a cell which has been genetically modified to express a specific Naᵥ1.9 channel (exogenous expression).

A further herein described method is an in vitro or ex vivo screening method for identifying an agent that modulates the Naᵥ1.9 channel activity, comprising:

a) exposing or contacting a tissue comprising nerves and Naᵥ1.9 channels, for example a skin-nerve preparation or an artificial tissue, to a test compound, and

b) detecting a decrease in response to a pain stimulus due to said exposition or contacting,

wherein said decrease identifies said test compound as an analgesic/antihyperalgic agent.

The contacting is typically performed by applying the compound at the tissue surface. The change in expression is preferably a decreased expression identifying the test compound as an inhibitor of the Naᵥ1.9 activity usable for preventing, alleviating or treating pain in a subject. The detection of a response to pain stimulus can be performed by recording an electrophysiological change in the nerve or neuron activity, typically an increase in the nerve or neuron activity in the absence of any analgesic compound or a decrease in the nerve or neuron activity when measured in presence of an analgesic compound according to the invention as herein described. The response can be detected using extracellular recording of nerve fibers in an ex vivo nerve-kin preparation, a calcium imaging platform to probe activity of cultured neurons in vitro and/or a patch-clamp electrophysiological rig to record the electrical activity of the neurons.

Preferably, this test compound demonstrates selectivity for the Naᵥ1.9 over other sodium channel subunits.

The present document also describes an in vivo method for identifying an analgesic/antihyperalgic agent, comprising:

a) administering a test compound to an animal, in particular an agent having activity in the previously herein described methods, and

b) detecting in said animal a decrease in response to a pain stimulus due to said administering and

wherein said animal preferably retains mechanical sensitivity in physiological condition and preferably also the ability to respond to non-nociceptive sensory stimuli (typically hot, cold and touch),

wherein said decrease identifies said test compound as an analgesic/antihyperalgic agent.

This method can further comprise additional steps of administering a test compound to a Naᵥ1.9 KO animal and comparing the reaction (response to a pain stimulus) of aid KO animal to that of the wildtype littermate.

Compounds when considered as having potential therapeutic value are subsequently analyzed using any standard in vitro assay or in vivo animal model for the disease indication known in the art. These compounds are considered as products of the invention and are usable as herein previously explained.

A further object of the invention is a kit comprising i) a product as herein described inhibiting the Naᵥ1.9 canal activity or a composition comprising such a product, preferably ii) at least one additional distinct active compound efficient against pain, and optionally iii) written instructions for using the kit.

According to a specific embodiment, the invention also relates to a kit that is suitable for the treatment by the methods herein described. These kits comprise i) a product as herein described inhibiting the Naᵥ1.9 canal activity or a composition comprising such a product, typically in the dosages herein indicated, and ii) a second composition containing an analgesic compound, preferably an opiate compound, in dosages generally lowered when compared to those classically prescribed, for a simultaneous, separate or sequential administration, in effective amounts according to the invention.

A further object of the invention relates to the use of a kit, as herein described, to prevent, alleviate or treat pain.

The figures and examples illustrate the invention without limiting its scope.

### LEGENDS TO THE FIGURES

FIG. 1: A) Nav1.9 concentrates in the flotillin-containing membrane fraction in rat and mouse DRG neurons. DRG lysates were fractionated using discontinuous optiprep gradients. Fraction 1 represents the top of the gradient. Gradients were fractionated, and equal amounts of proteins from corresponding fractions were separated by SDS-PAGE. Western blots were probed for Nav1.9, β-actin, flotillin, and TfR1. Flotillin, which is a marker for lipid raft membranes, was used as a marker for the lipid raft fraction, while TfR1 and β-actin were used to mark the heavy membrane fractions. These results are representative of at least three independent experiments.

B) Upon exposure to a cocktail of inflammatory mediators, Nav1.9 is partitioned to non-raft domain (right panel) compared to sham treated mouse DRG (left panel).

C) Quantification of signal from at least 3 independent experiments shows that inflammatory cocktail specifically sort out Nav1.9 from lipid raft without affecting flotillin distribution.

FIG. 2: A) Time course of tactile hyperalgesia measured with von Frey filaments in wild type and Nav1.9 KO mice, intraplantarly injected with 20 μl of methyl-beta-cyclodextrin, (40 mM); or saline solution. Intraperitoneal injection of the anti-inflammatory molecule ibuprofen (75 mg/kg) does not modify methyl-beta-cyclodextrin-induced tactile hyperalgia.

B) Time course of tactile hyperalgesia measured with von Frey filaments in wild type and Nav1.9 KO mice, intraplantarly injected with 20 μl of methyl-beta-cyclodextrin, (40 mM); or alpha-cyclodextrin (40 mM) solutions.

C) MβCD injection does not induce inflammatory process since intraperitoneal injection of the anti-inflammatory molecule ibuprofen (75 mg/kg) does not reverse allodynic phenotype (green wt; blue KO).

FIG. 3: Time course of tactile hyperalgesia measured with von Frey filaments in mice intraplantarly injected with 20 μl of methyl-beta-cyclodextrin, (40 mM); or alpha-cyclodextrin (40 mM) solution. Alphα-cyclodextrin does not induce tactile hyperalgia.

FIG. 4: A) and B) show the time course of tactile hyperalgesia measured with von Frey filaments in mice intraplantarly injected with 20 μl of 2% lambdα-carrageenan, intraperitoneal injection of ibuprofen reverse lambdα-carrageenan-induced tactile hyperalgesia. Intraplantar injection of cholesterol complexed with methyl-beta-cyclodextrin (soluble cholesterol, 5; 6 mM of cholesterol) also reverses lambdα-carrageenan-induced tactile hyperalgesia to control levels (saline injected animals). Cholesterol does not modify mechanical sensibility in control non-inflamed animals (black).

FIG. 5: In non-inflamed animals, intraplantar injection of soluble cholesterol does not change mechanical threshold of wild type and Nav1.9 KO mice in physiological non inflamed conditions.

FIG. 6: Inflammation reduces cholesterol level in skin and neuron membrane

A) Cholesterol level in skin tissue was quantified before and during the time course of persistent inflammation induced by intra plantar injection of λ-carrageenan. Cholesterol level is significantly reduced during the first couple of hours after inflammation onset.

B) Inflammatory mediators decrease cholesterol level of sensory neurons in culture (n=4).

FIG. 7: Peri-articular injection of cholesterol improves chronic pain behavioral.

In arthritic mice, periarticular injection of cholesterol (Blue) improved all posture parameters examined compared to animal injected with saline solution (red).

A-B) inflamed limbs, once injected with cholesterol (20 μl of a 5.6 mM solution, blues line) bear more weight than limbs injected with saline solution (20 μl).

C) As a consequence of severe painful limb, mice put less weight to their inflamed paw, and more to their tail (not shown). Cholesterol injection reduces the weight put on tail compared to saline injected mice.

FIG. 8: Transdermal delivery of cholesterol relieves carrageenan induced pain

Application of a hydroxycellulose gel containing 5.6 mM of cholesterol for 1.5 hrs abolishes mechanical allodynia compared to control gel with hydroxy cellulose alone.

FIG. 9: Transdermal delivery of cholesterol is an efficient pain killer for chronic arthritis pain

Application of a hydroxycellulose gel containing cholesterol (28 mM, black triangle), rescue chronic pain of arthritic mice for several hours. Application of a 5% ibuprofen hydroxycellulose gel lessens mechanical pain to a much lower extend (grey circle) than cholesterol. Mice treated with hydroxycellulose gel only do not exhibit modification of their painfull phenotype during the time course of the experiment (open circles).

FIG. 10: Transdermal delivery is a possible topic route of administration for human pathologies

Application of a hydroxycellulose gel containing cholesterol results in an increase of cholesterol content in human dermis compartment after a 12 h (not shown) or 24 h application period. Carbopol gel allows epidermis delivery of cholesterol in human skin.

FIG. 11: cholesterol modulates Nav1.9 activity and is able to reverse inflammatory cocktail effect on channel's activation properties.

A) Inflammatory cocktail shifts Nav1.9 activation curve to more hyperpolarised potentials (blue curve) compared to control neuron (black curve). Such shift accounts for enhanced excitability of neurons upon inflammation. Cholesterol supply reverses the shift (red curve).

B) Cholesterol depletion by MβCD (red curve) or cholesterol oxidation by cholesterol oxidase (blue curve) induces the same shift as inflammatory cocktail.

C) Cholesterol per se (blue curve) does not modify activation properties of Nav1.9.

### EXAMPLES

Comparing pain phenotype in wild type (wt) or knocked-out (KO) mice, inventors established the proof of concept that disturbing lipid raft architecture by cholesterol uptake causes Nav1.9 activation which contributes to pain signalling.

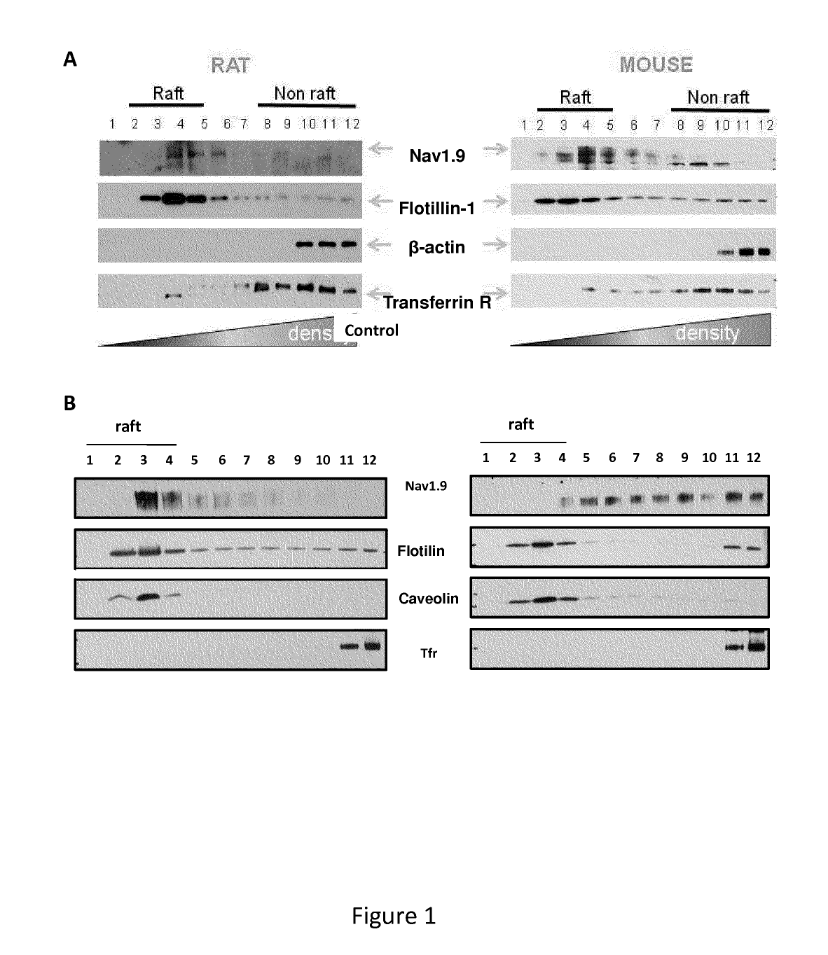

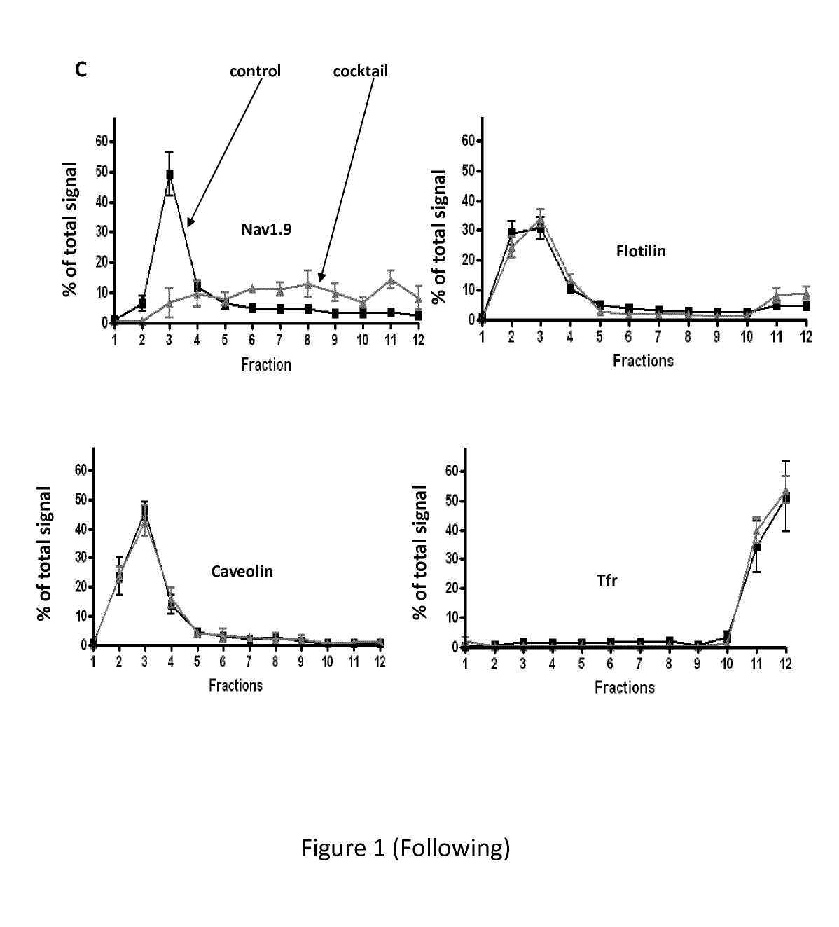

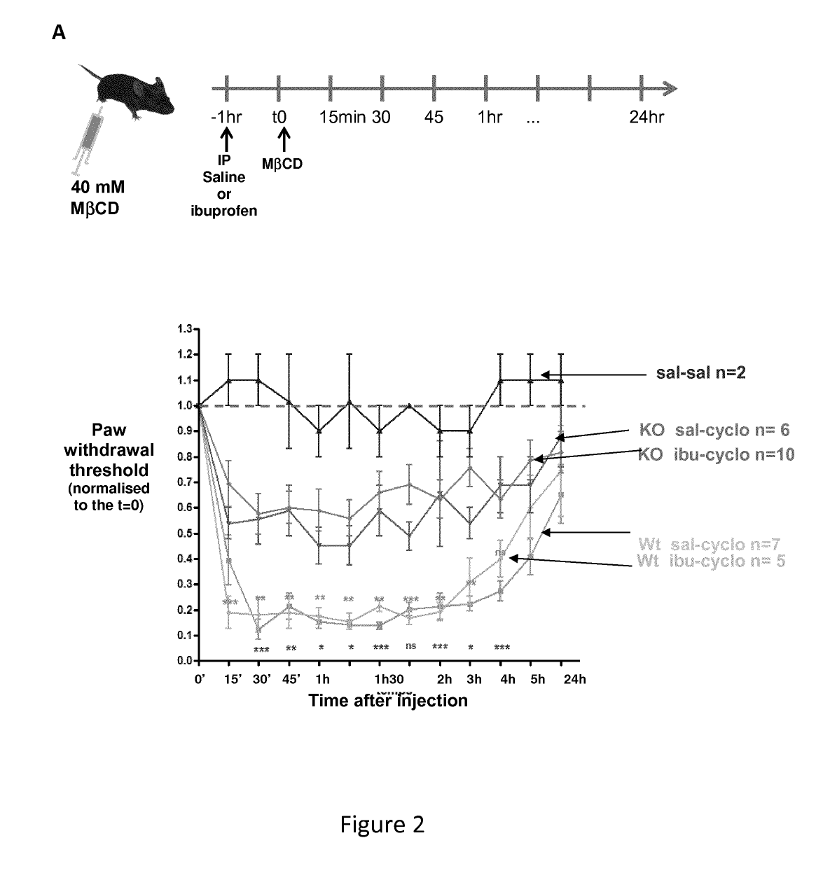

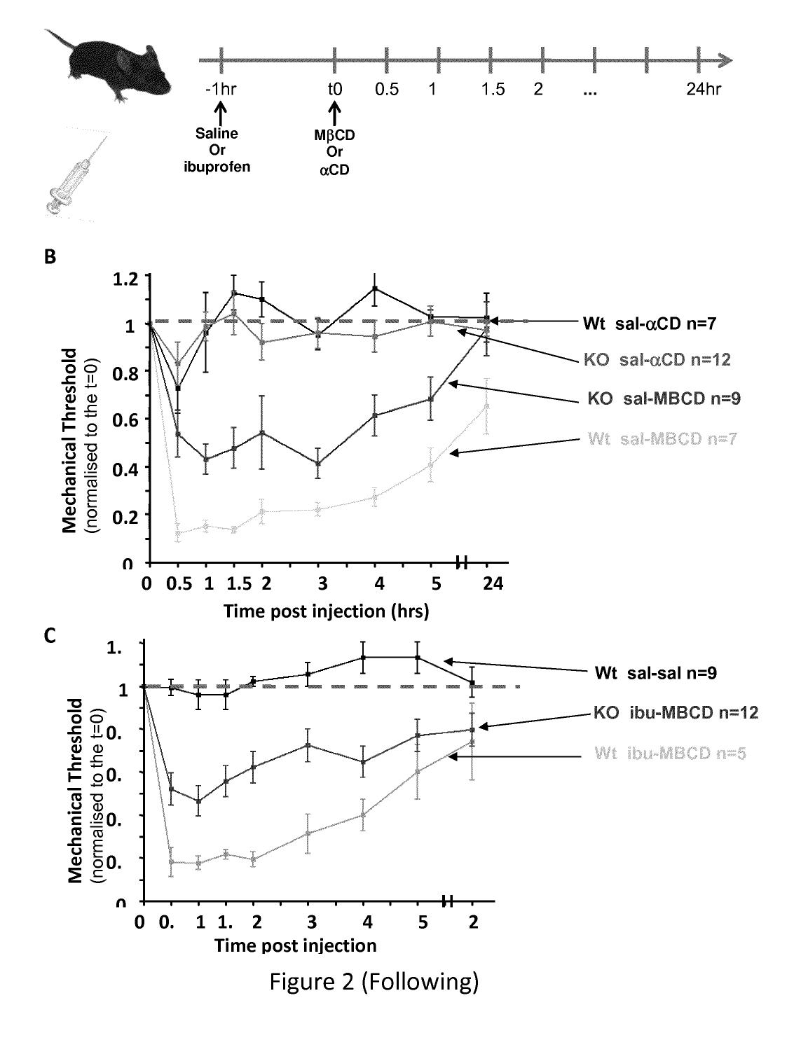

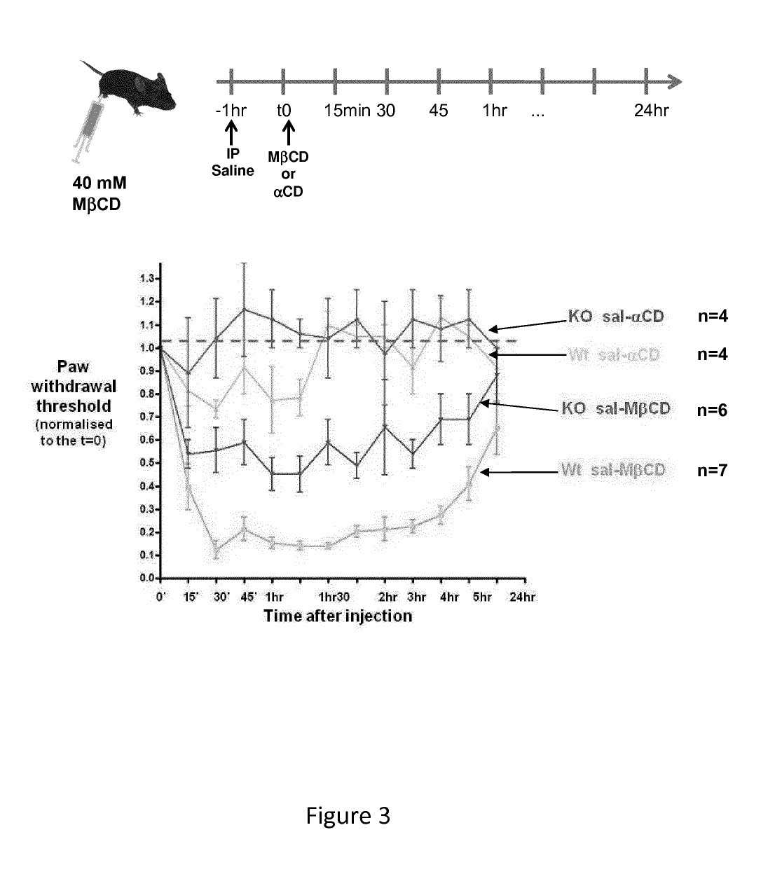

Inventors demonstrated that Naᵥ1.9 channels are localised in lipid raft microdomains at the cell membrane of nociceptive neurons of dorsal root ganglia (FIG. 1A), and that a cocktail of inflammatory molecules partitions Naᵥ1.9 out of lipid raft domains (FIG. 1B-C). As herein explained, inventors used methyl-beta-cyclodextrin (MBCD) to selectively extract cholesterol from the plasma membrane. They showed that intra plantar injection of 20 μl of a 40 mM solution of MBCD induces a strong mechanical allodynia as measured with the von Frey filament test. This MBCD-induced allodynia is markedly reduced in Nav1.9 KO animals. Inventors showed that this allodynia is not due to an inflammatory process since intrα-peritoneal injection of ibuprofen (75 mg/kg) did not change mechanical threshold neither in wild type nor in Nav1.9 KO animals (FIGS. 2A, 2C). Moreover, similar injection of 40 mM of α-cyclodextrin (α-CD), an inactive analogue of methyl-beta-cyclodextrin that cannot extract cholesterol from membrane, did not induce mechanical allodynia (both in wild type and Naᵥ1.9 KO animals; FIG. 2B; FIG. 3).

Taken all together these results show that MBCD-induced cholesterol efflux activates Naᵥ1.9 channels and results in mechanical allodynia.

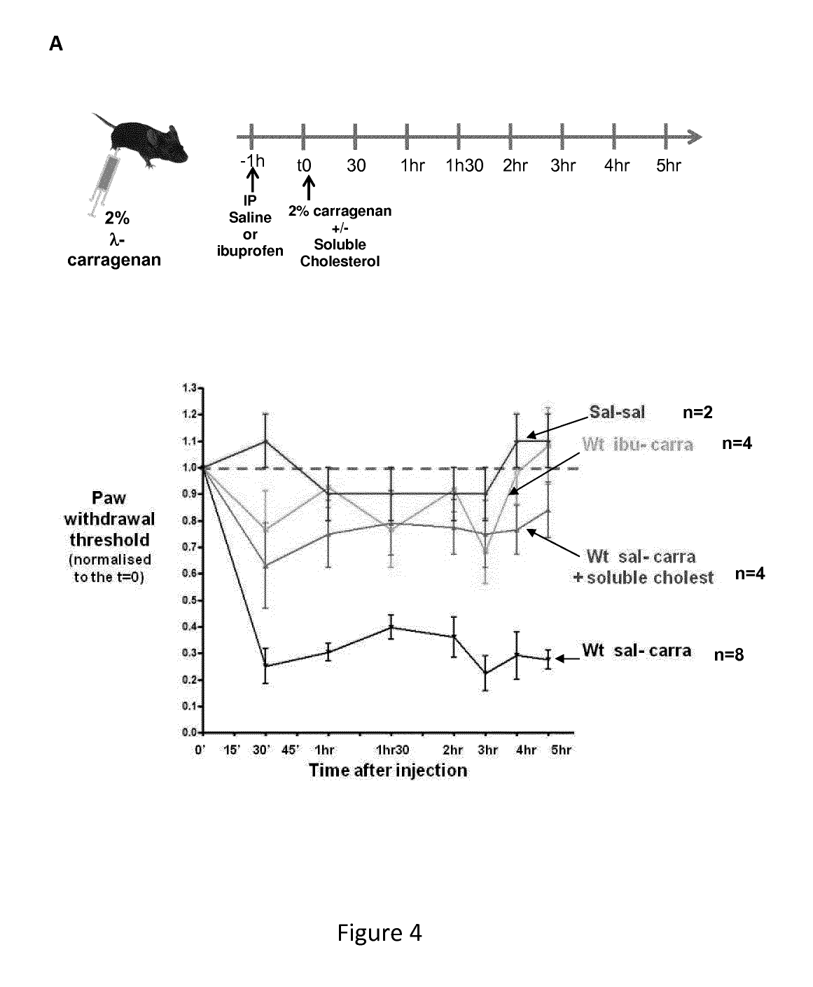

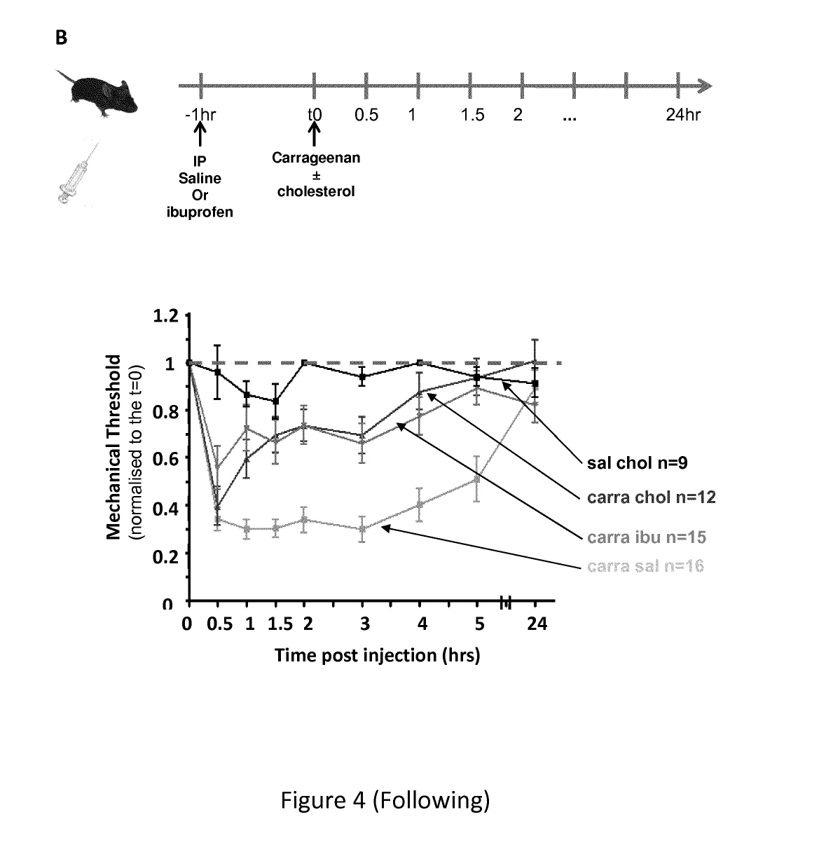

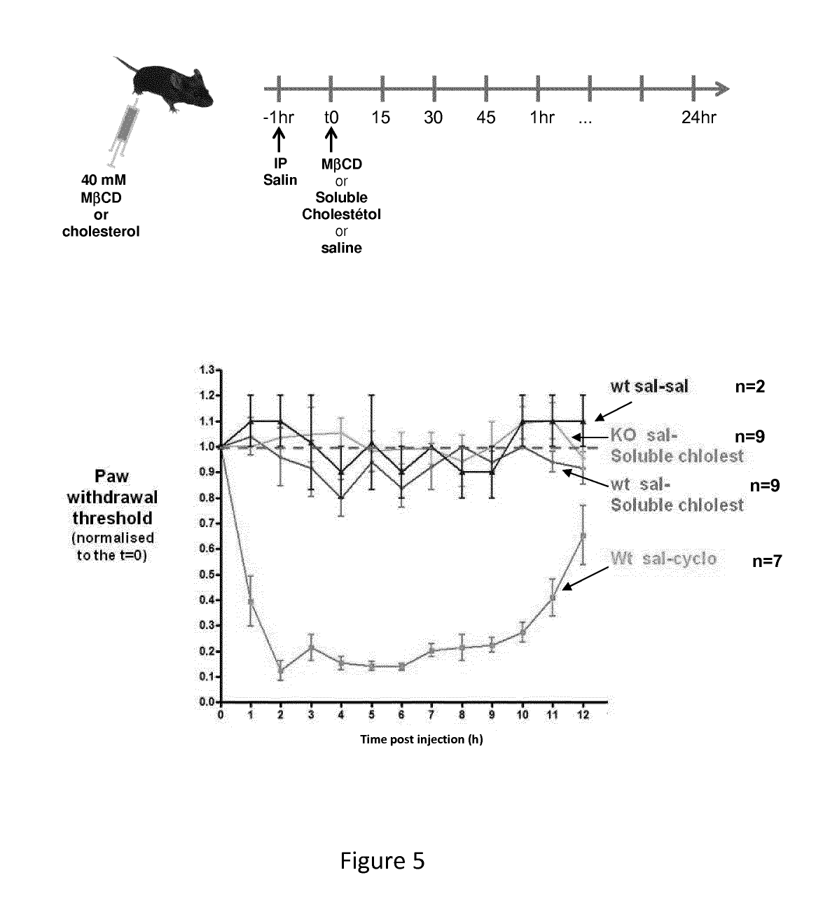

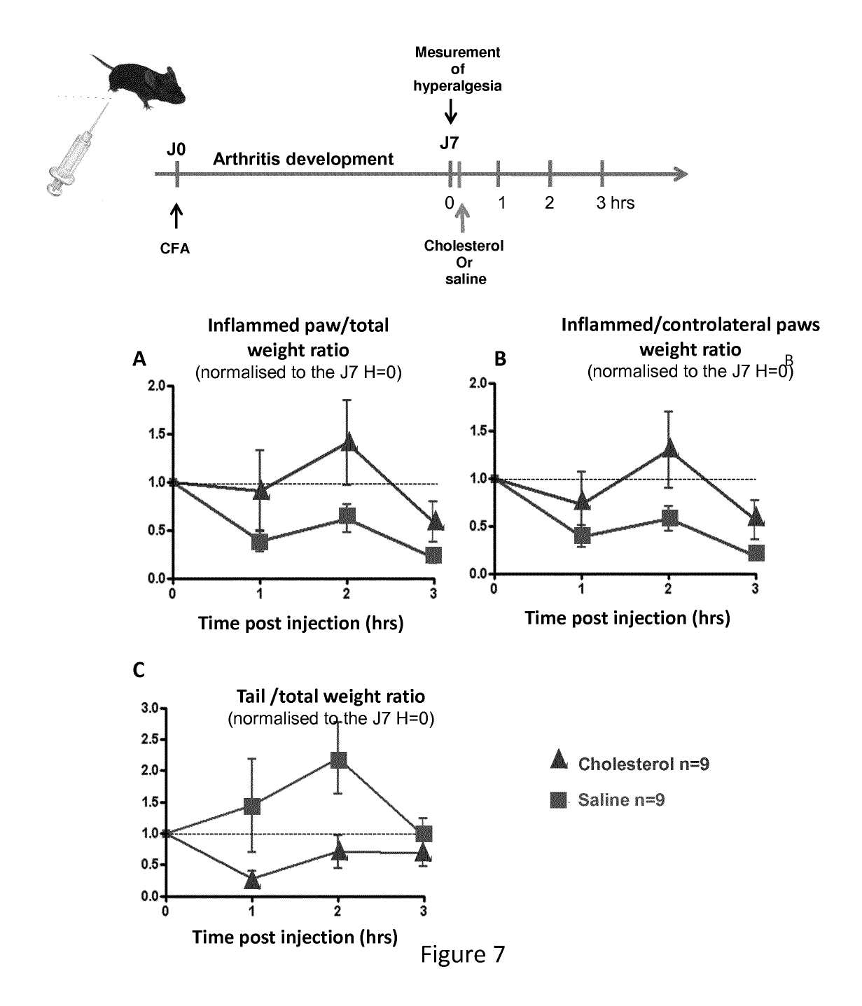

Conversely, inventors showed that topic application of cholesterol inhibits inflammatory evoked pain (FIG. 4 A-B), and to improve behavioural parameter of arthritic mice (FIG. 7), without changing physiological mechanical threshold (n=9 both in wild type and Naᵥ1.9 KO animals; FIGS. 4B-5).

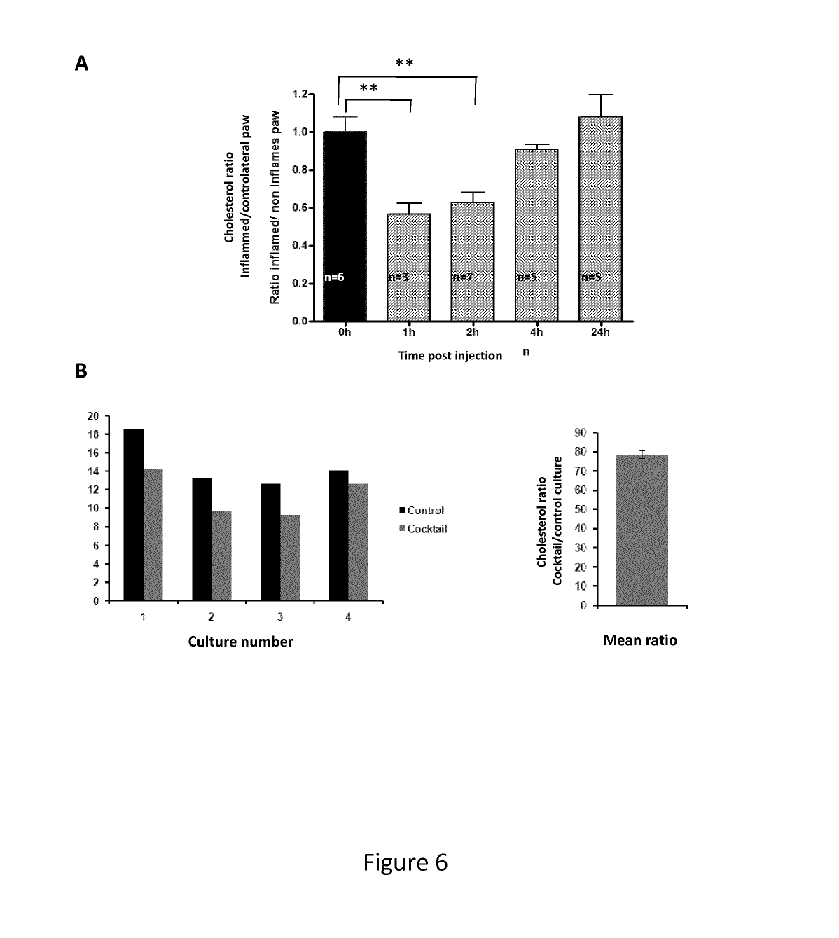

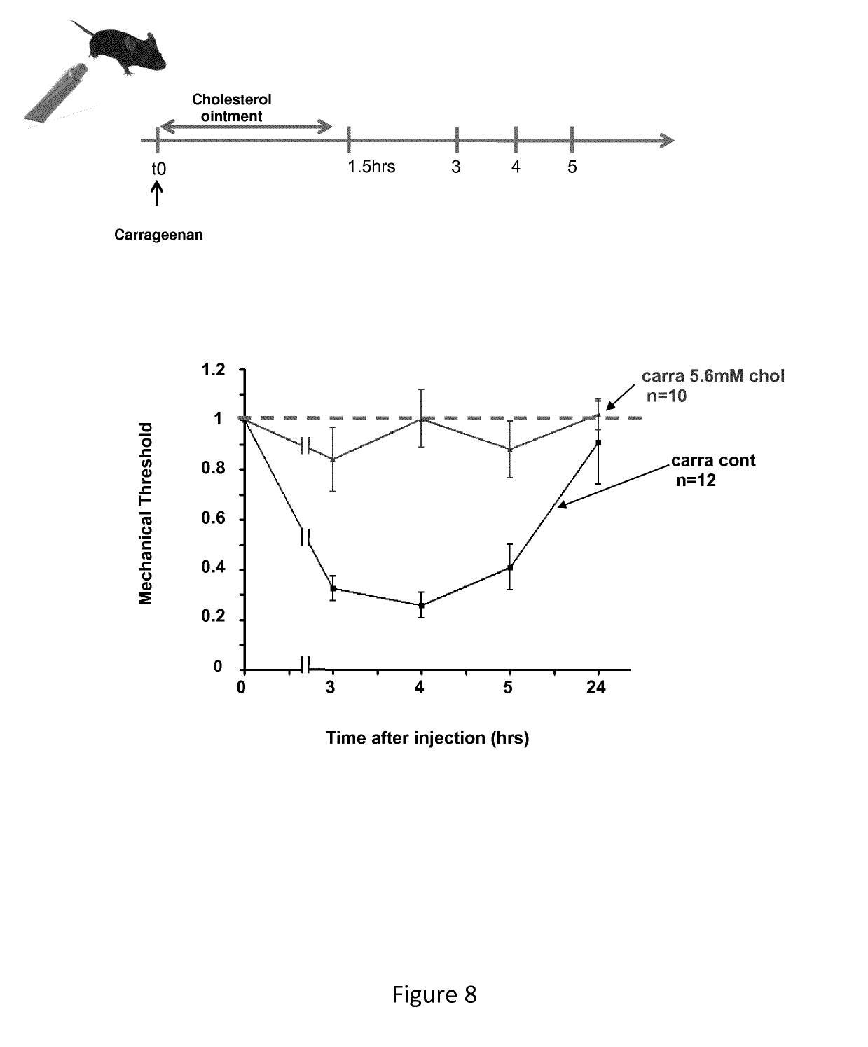

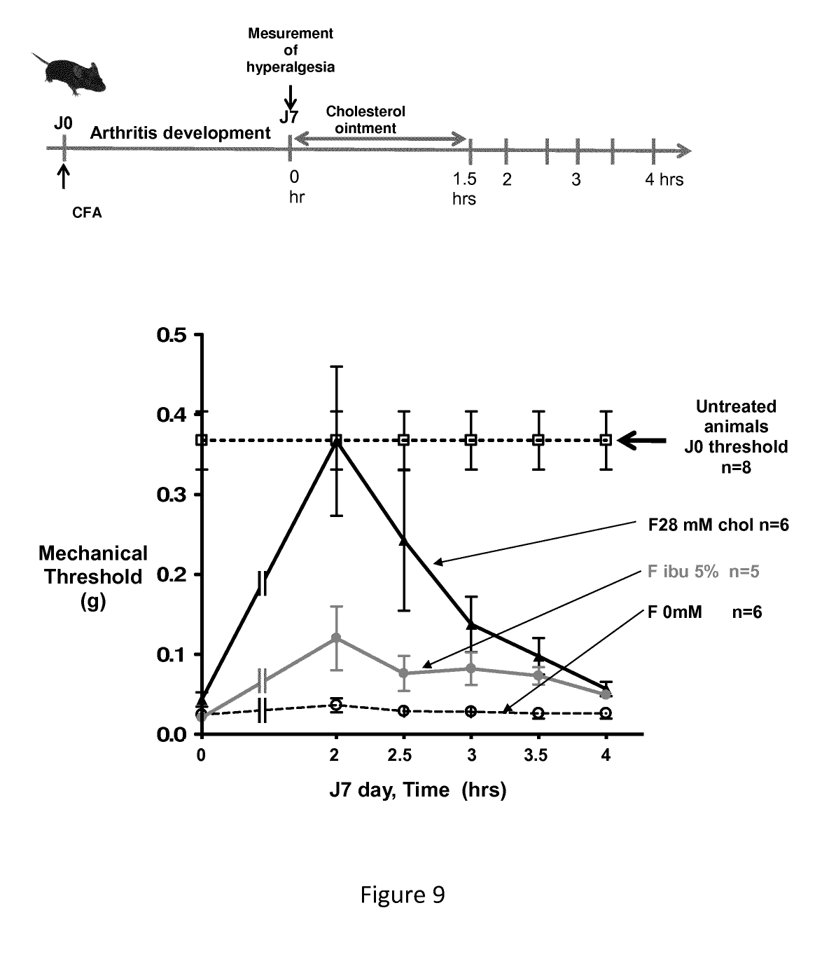

Inventors demonstrated that during inflammation, induced by intraplantar carragenine injection, the level of endogenous cholesterol is decreased (FIG. 6A) and that in vitro incubation of DRG neuron with a cocktail of inflammatory mediators also reduces membrane cholesterol content of treated neurons (FIG. 6B). This result explains the rationale of a supply of cholesterol to alleviate pain. Inventors demonstrated that transcutaneous delivery of cholesterol kills carragenin induced pain (FIG. 8), and is much efficient to treat arthritic inflammatory pain than a 5% ibuprofen gel (FIG. 9).

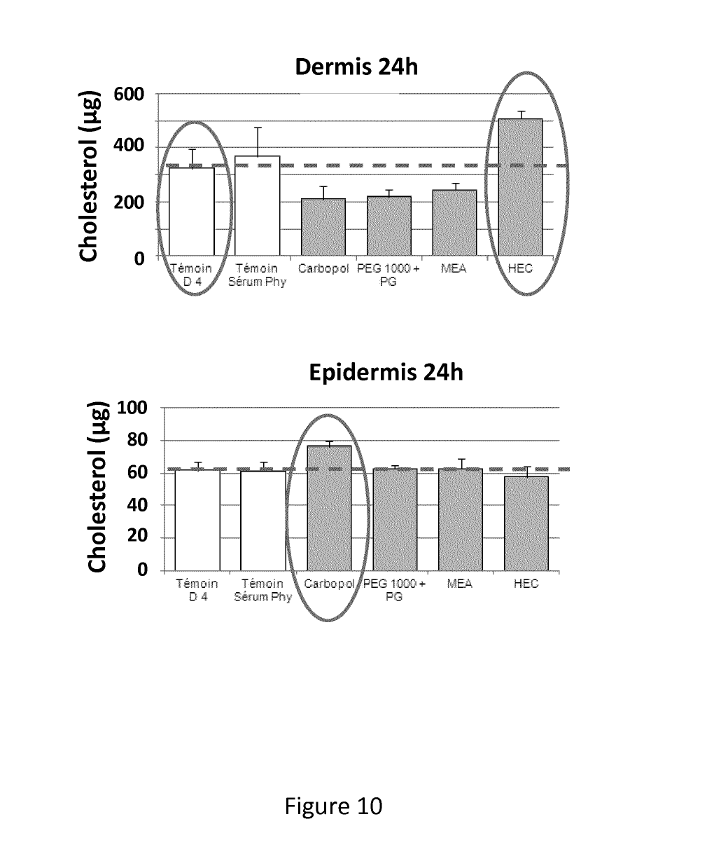

Inventors demonstrated that transcutaneous delivery is also possible in the context of a human medical treatment (FIG. 10).

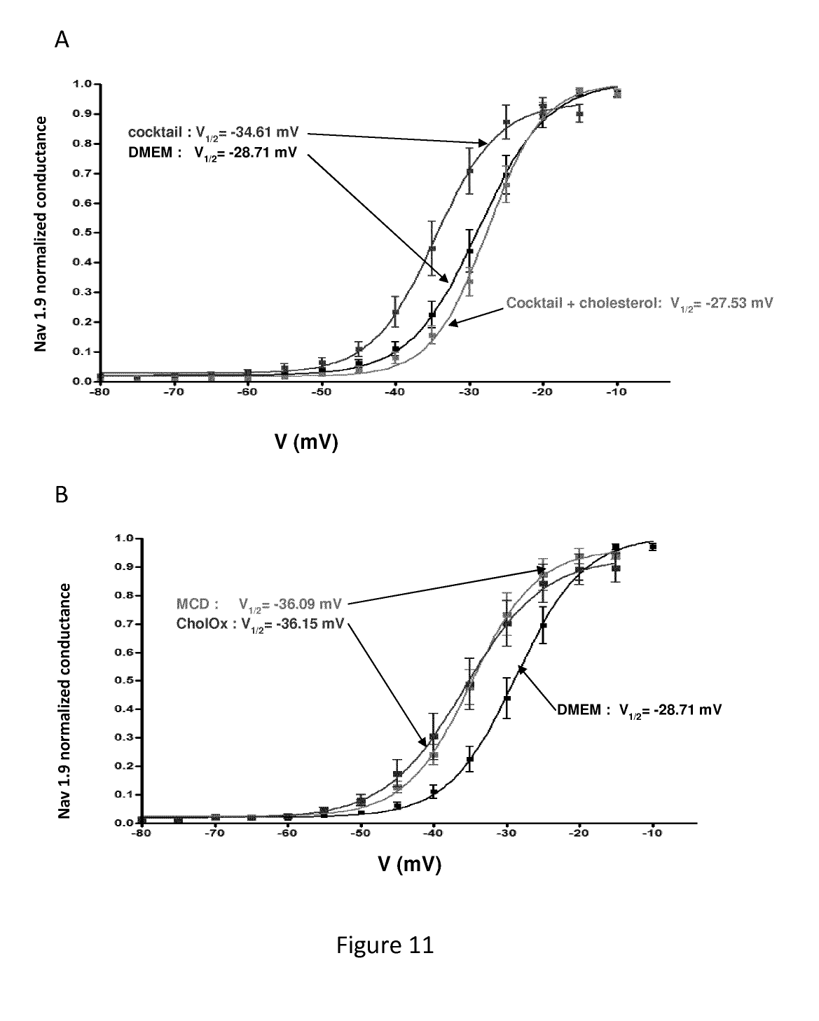

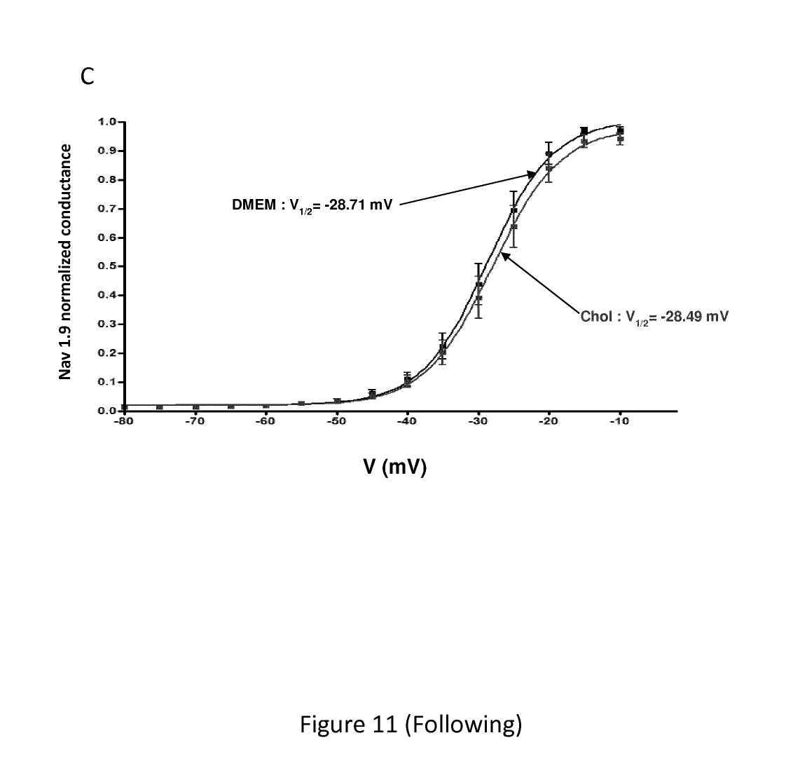

Inventors demonstrated that cholesterol depletion by MBCD or cholesterol oxidase incubation shift Naᵥ1.9 activation curves to hyperpolarised potential in the same range of amplitude than inflammatory cocktail. Moreover, cholesterol supply reverses this shift of activation (FIG. 11).

Materials & Methods

All animals were used in accordance with the European Community guiding in the care and use of animals (86/609/CEE). They were housed under controlled environmental conditions and kept under a 12/12 h light/dark cycle, with food and water ad libitum.

Raft Gradient Fraction:

Lipid-rich plasma membrane domains were isolated, as detergent-resistant membranes (DRMs), principally by flotation from a dense solution through a discontinuous or continuous density gradient containing the non-ionic detergent Triton X-100.

Freshly dissociated DRG from one mouse were homogenized in 140 μl of lysis buffer with 0.5% triton X-100 and 1% proteinase inhibitor cocktail (Roche). The homogenate centrifuged at 1,000 g for 10 min at 4° C. and the supernatants were incubated 1 h on ice, and then subjected to density gradient ultracentrifugation along OptiPrep™ step density gradients.

TLS-55 ultracentrifuge tubes were filled from the bottom to the top as follow: 140 μl of the supernatant was mixed with 280 μl of 60% Optiprep™. To this, 1.6 ml of 30% Optiprep™ (1:1 mix of 60% optiprep solution with lysis buffer) was overlaid, followed by 200 μl of 0% Optiprep™ (lysis buffer only). The tubes were centrifuged at 55 00 rpm (200,000 g) for 4 h at 4° C. Twelve fractions of 150 μl each were gently removed from the top of the tube and individually aliquoted. Fractions were numbered from low (n° 1) to high (n° 12) density fractions. The fractions were stored at −80° C. for up to 6 months.

Fractions were further analysed by western blot. As protein concentration does not provide a true baseline parameter for comparison across fractions, equal sample volume loading was used for the Western blot. This loading method accounts for differential separation of proteins into specific fractions following density ultracentrifugation. From each fraction, an equal sample volume (30 μl) was separated into 4-12% SDS polyacrylamide gels. Protein was transferred onto nitrocellulose membranes. Membranes were incubated with 1:1,000 dilution of an antibody to flotillin 1 (Sigma) to identify the low-density raft fractions. Membranes were additionally probed with a 1:500 dilution of an antibody to UR (Invitrogen) and with a 1:10000 β-actin (Sigma) to identify non raft fractions. Separately, membranes were probed with a 1:2000 dilution of the Nav1.9 antibody produced in our laboratory. Roche POD Western Chemiluminescent kits were used for protein detection.

For drug incubation DRG were collected in inflammatory cocktail or in DMEM, and incubated for 15 min at 37° C. After several washes in lysis buffer they were proceeded as describe above.

Cyclodextrin and Cholesterol Manipulations:

Methyl βcyclodextrin or α-cyclodextrin (both from sigma) were diluted in saline buffer prior to injection. Methyl βcyclodextrin/cholesterol complex (40 mg of cholesterol/g of complex) also called soluble cholesterol was purchased (from Sigma). Unless otherwise states, all cyclodextrin or cyclodextrin/cholesterol complexes were injected at a 40 mM concentration of cyclodextrin corresponding to 5.6 mM of cholesterol in the complex.

Carrageenan Model:

Persistent paw inflammation was induced by intraplantar injection of 20 μl of 2% λ-carrageenan (Sigma) in mice. Control animal received only vehicle saline solution (0.9% NaCl).

1 hr prior to carrageenan injection, animals received intra peritoneal injection of either saline or ibuprofen (75 mg/kg in saline solution).

Complete Freund's Adjuvent (CFA) Induced Chronic Inflammation:

Chronic inflammation (monoarthritis model) was induced under 2% isoflurane anesthesia by two subcutaneous injections of 15 μl around the tibio-tarsal joint of mice with complete Freund adjuvant (CFA), containing 5 μg/μl heat-killed Mycobacterium butyricum (Becton, Dickinson and cie).

Behavioral Assays

Von Frey Test

Mechanical pain threshold was assessed using von Frey filaments (BioSeb) calibrated from 0.008 to 4.000 g. The filaments were applied five times each, in order of increasing forces, and pressed perpendicularly to the plantar surface of the hindpaw until they bent. The first filament which evoked at least 3/5 response was assigned as the pain threshold in grams.

Dynamic Weight Bearing

The animals' weight distribution on the four limbs was assessed using the dynamic weight bearing test (BioSeb). This new incapacitance test consists in a continuous measurement of all pressure points applied by a freely moving animal, allowing a quantitative evaluation of the weight imbalance caused by paw mechanical allodynia. Mice were placed in an 11×11×22 cm cage with a 44×44 sensor cells grid on the floor. The pressure applied on sensor cells by the animal's paws is recorded at a 10 Hz frequency over a 5 min period. Pressure and surface detection thresholds were determined automatically for each animal by the Dynamic weight bearing 1.3.2h software (Bioseb). After the manual attribution of each pressure point to the corresponding paw, the mean weight applied on each paw is calculated. Unilateral pain is finally evaluated trough the ipsi/contralateral hindpaws weight ratio, the weight applied on forepaws, and the percentage of time spent raising ipsilateral hindpaw.

DRG Neurons Culture

Dissociation of DRG neurons has been previously described (Coste et al., 2004; Coste et al., 2007). In brief, thoraco-lumbar DRG neurons were excised and incubated in enzyme solution containing 2 mg/ml of collagenase IA (Sigma) for 45 min at 37° C. before trituration in Hanks' medium (Life Technologies). Culture medium was Dulbecco's modified Eagle's medium (DMEM, Life Technologies) supplemented with 10% heat-inactivated fetal calf serum, 50 U/ml penicillin-streptomycin, 2 mML-glutamine, 25 mM glucose, 2 ng/ml glial-derived neurotrophic factor (GDNF) (all from Life Technologies), and 25 ng/ml nerve growth factor (NGF, Millipore). Cells were seeded on 35 mm cultures dishes (Nunc) coated with 2 mg/ml laminin (Sigma) and maintained in a humidified atmosphere (5% CO₂, 37° C.) for 16-24 h before further cholesterol manipulations or electrophysiological recording.

Cholesterol Quantification

Inflamed and controlateral skin paw, were dissected with biopsy punch of 3.5 mm diameter.

For cultures of DRG neurons, after incubation with inflammatory cocktail, cells were washed 3 times with D-PBS at 37° C. (Life Technologies) then dissociated with 5 min, 0.05% trypsin-EDTA incubation. Cells were next centrifugated for 5 min at 3300 rpm then pellet was resuspended in 50 μl lysis buffer (150 mM NaCl, 20 mM Tris pH 7.5, 2 mM EDTA, 1% Triton X-100 with protease inhibitor cocktail (Roche)) and homogenized by vortex and sonication. Cell lysate was centrifuged at 10 000 rpm for 2 min. 10 μl of supernatant were used to determine protein concentration by BCA protein assay kit (Pierce). Lipids were extracted on the remaining supernatant (40 μl)

Determination of cholesterol content was performed with an enzymatic fluorimetric assay (Biovision) according to the manufacturer instructions.

Drugs and Chemicals

Inflammatory cocktail contained 50 nM bradykinin (BK), 500 nM prostaglandin-E2 (PGE2), 1 μM histamine (His), 500 nM norepinephrine (NE), and 2 μM ATP (all from Sigma-Aldrich).

Water soluble cholesterol refers to a complex of MβCD-cholesterol and was purchased from sigma Aldrich, MP bio or cyclodextrin CT. The molar ratio between MβCD and cholesterol is 7.14:1, and concentrations cited in this article refer to MβCD concentration.

Other chemicals: Hydroxyethyl cellulose MβCD, and αCD were from Sigma Aldrich. Cholesterol oxidase was from Calbiochem.

Electrophysiology

Patch pipettes had resistances of 3-3.5 MΩ for whole-cell voltage clamp recordings. Intracellular solution used in this study contained: 130 CsCl, 10 HEPES, 8 NaCl, 5 EGTA, 2.4 CaCl₂, 1 MgCl₂ (pH 7.3, 298 mOsm/l). For voltage clamp recording of Na⁺ currents, extracellular solution had a standard driving force for Na⁺ (in mM): 130 NaCl, 10 glucose, 3 KCl, 1 MgCl₂, 10 HEPES, 2.5 CaCl₂(pH 7.35, 293 mOsm/l). Extracellular solution contained 500 nM tetrodotoxin (TTX) as well as 1 mM amiloride and 50 μM La³⁺ in order to block Ca²⁺ currents without altering NaN/Nav1.9 properties (Coste et al., 2007). All experiments were performed at room temperature and chemicals were obtained from Sigma-Aldrich (except TTX, from Abcam). Extracellular media were exchanged using a gravity-fed bath perfusion system at a flow rate of 2-5 ml/min, while bath solution was removed by continuous suction. Recycling was used to reduce the amount of TTX used. Stock solutions of TTX (0.1 mM) and La³⁺ (1M) were prepared in water. Stock solution of amiloride hydrochloride (1M) was made in dimethylsulfoxyde (final concentration, 0.1%).

For drugs application, DRG cultures were incubated for 15 min at 37° C. in DMEM containing freshly prepared MβCD (20 mM), water soluble cholesterol (20 mM), or cholesterol oxidase (4 U/ml). The inflammatory cocktail was incubated in extracellular solution for at least 20 min at room temperature (RT).

Data Acquisition and Analysis

Data were acquired with an Axopatch 200B amplifier (Axon Instruments), filtered at 2 kHz, and digitally sampled at 20 kHz using PCLAMP 10 software. Currents were leak subtracted using a P/6 protocol. Voltage errors were minimized using 70-85% series resistance compensation. Whole-cell recording were begun 1 min after achieving the whole-cell configuration, to allow Cs⁺ to equilibrate and to allow Nav1.9 channels to recover from slow inactivation. Conductance-voltage curves were calculated from the peak current according to the equation G=I/(V−Eᵣₑᵥ), where V is the test pulse potential and Eᵣₑᵥ the reversal potential calculated according to the Nernst equation. The activation curve (G-V) was fitted using the Boltzmann function: G/Gₘₐₓ=1/(1+exp[(V_{1/2}−V)/k]), where G/Gₘₐₓ is the normalized conductance, V_{1/2} is the potential of half-maximum channel activation, and k is the steepness factor.

PRISM 4.0 (GraphPad) software was used to perform linear and nonlinear fitting of data. Results are presented as mean±SEM and n represents the number of cells examined. Statistical analysis used Student's t test and P<0.05 was considered statistically significant.

### CONCLUSION

The inventors demonstrate that supply of a lipid inhibiting the Naᵥ1.9 channel activity, in particular cholesterol, is a new pain killer drug strategy that alleviates, lessens, and/or prevents persistent and/or chronic pain. Cholesterol in particular modulates the activity of Nav1.9 channel, reducing nociceptors excitability. These new antalgic/antihyperalgesic molecules act through different molecular pathway than NSAIDs, opioid molecule and are more potent than ibuprofen to reduce chronic arthritic pain. These results open the possibility to design novel powerful pain killer strategy by combination of one or several of the herein described molecules.

1-15. (canceled)

16. A method for alleviating or treating pain in a subject comprising a step of administering to the subject, or exposing said subject, to an effective amount of a lipid inhibiting Nav1.9 channel activity.

17. The method according to claim 16, wherein the lipid inhibiting the Naᵥ1.9 channel activity is selected from cholesterol, soluble cholesterol, a sphingolipid and any combination thereof.

18. The method according to claim 16, wherein the pain is an acute pain, a subacute pain or a chronic pain.

19. The method according to claim 16, wherein the pain is allodynia or hyperalgesia.

20. The method according to claim 16, wherein the pain is selected from an inflammatory pain, a mechanical heat or cold hypersensitivity, a visceral pain, an enteric pain, a somatic pain or a trigeminal neuralgia.

21. The method according to claim 18, wherein said pain is associated with a gastrointestinal syndrome.

22. The method according to claim 16, wherein said pain is a pain sensitive to steroidal anti-inflammatory drug (SAID), non-steroidal anti-inflammatory drug (NSAID) or opioid, and wherein said pain is to be treated with a combination of said lipid together with a reduced amount of SAID, NSAID or opioid when compared to the administration of SAID, NSAID or opioid alone.

23. The method according to claim 16, wherein the subject is resistant to a drug selected from SAID, NSAID and opioid, or suffers from gastrointestinal and/or renal adverse effects induced by said drug.

24. The method according to claim 16, wherein the lipid is administered to the subject through cutaneous, subcutaneous. intrathecal, intra spinal, dermic, transdermic, ocular or rectal routes.

25. The method according to claim 16, wherein the subject is a vertebrate.

26. A composition comprising a lipid inhibiting the Nav1.9 channel activity as an active ingredient and a pharmaceutically acceptable carrier.

27. The composition according to claim 26, wherein said composition further comprises at least one additional active compound.

28. The composition according to claim 27, wherein said at least one additional active compound is an inhibitor of the degradation of a lipid inhibiting the Naᵥ1.9 channel activity.

29. The composition according to claim 28, wherein said inhibitor is a cholesterol oxidase inhibitor or a sphingomyelinase enzyme inhibitor.

30. The composition according to claim 26, wherein said composition is a local analgesic and/or anti-hyperalgesic composition.

31. A kit comprising i) a lipid inhibiting the Nav1.9 channel activity or a composition comprising such a lipid according to claim 26, and ii) at least one additional distinct active compound efficient against pain.
